(19) United States
(12) Patent Application Publication
(10) Pub. No.: US 2023/0226070 A1
(43) Pub. Date: Jul. 20, 2023

(54) TREATMENT OF THROMBOSIS AND ASSOCIATED DISORDERS WITH AN ANTI-PLATELET AGENT.

(71) Applicants: THE HEART RESEARCH INSTITUTE LTD, Newtown, New South Wales (AU); THE UNIVERSITY OF SYDNEY, SYDNEY NEW SOUTH WALES (AU)

(72) Inventors: Shaun JACKSON, Durham, CT (US); Simone SCHOENWAELDER, Newtown, New South Wales (AU)

(21) Appl. No.: 17/998,586

(22) PCT Filed: May 14, 2021
(86) PCT No.: PCT/AU2021/050454

§ 371 Date: Nov. 11, 2022

(30) Foreign Application Priority Data
May 14, 2020 (AU) 2020901558

(51) Int. Cl. A61K 31/5377; A61K 31/4709; A61K 38/48; A61P 7/02

(52) U.S. Cl. CPC A61K 31/5377; A61K 31/4709; A61K 38/482; A61P 7/02

(57) ABSTRACT

The present disclosure relates to the use of anti-platelet agents for the treatment of thrombosis and related conditions in a subject. The anti-platelet agent, preferably a phosphoinositide 3-kinase beta (PI3Kβ) inhibitor such as TGX221 or AZD6482, may be administered alone or in combination with a thrombolytic agent, preferably recombinant tissue plasminogen activator (rtPA), and/or an anticoagulant agent, preferably argatroban.

### CROSS-REFERENCE TO RELATED APPLICATIONS

The present application claims priority from Australian Provisional Patent Application No 2020901558 filed on 14 May 2020, the content of which is incorporated herein by reference in its entirety.

### TECHNICAL FIELD

The present disclosure relates to the field of therapeutic and prophylactic treatments of thrombosis or a disease or condition resulting from or associated with thrombosis. The present disclosure provides new therapeutic and prophylactic treatments of thrombosis and related conditions in a subject, comprising administering an anti-platelet agent to the subject. The anti-platelet agent may be administered alone or in combination with a thrombolytic agent and/or an anticoagulant.

### BACKGROUND

We are becoming increasingly aware of a growing number of clinical incidences of thrombosis resulting from a wider variety of causes.

The process of thrombosis is a complex interplay between clotting factors within the blood as well as one of the cellular components, platelets. These cellular fragments are involved in the pathology of thrombotic disorders and inhibition of platelet function is protective in conditions such as myocardial infarction and stroke. One of the biochemical pathways within platelets is known as PI 3-kinase beta (PI3Kβ). A pharmacological antagonist of this pathway (AZD6482) has been demonstrated previously to block platelet function. Current existing antiplatelet therapies (aspirin and clopidogrel, among others), are associated with bleeding complications, some of which may be life threatening.

### SUMMARY

The present disclosure is based on the inventors' surprising finding that anti-platelet agents can enhance the efficacy of known treatments for thrombosis and associated conditions. Thus, the present disclosure provides adjunct therapies for the treatment of thrombosis or a disease or condition resulting from or associated with thrombosis, comprising the administration of an anti-platelet agent.

In one aspect, the present disclosure provides a method of treating thrombosis or a disease or condition resulting from or associated with thrombosis, in a subject in need thereof, the method comprising administering to the subject an anti-platelet agent.

In one aspect, the present disclosure provides a method of treating thrombosis or a disease or condition resulting from or associated with thrombosis, in a subject in need thereof, the method comprising administering to the subject an anti-platelet agent, and wherein the method further comprises the simultaneous, sequential or separate administration of a thrombolytic agent and/or an anti-coagulant.

In another aspect, the present disclosure provides the use of an anti-platelet agent in the manufacture of a medicament for treating thrombosis or a disease or condition resulting from or associated with thrombosis in a subject in need thereof.

In another aspect, the present disclosure provides the use of an anti-platelet agent in the manufacture of a medicament for treating thrombosis or a disease or condition resulting from or associated with thrombosis in a subject in need thereof, wherein the anti-platelet agent is prepared for simultaneous, sequential or separate administration with a thrombolytic agent and/or an anti-coagulant.

In another aspect, the present disclosure provides an anti-platelet agent for use in treating thrombosis or a disease or condition resulting from or associated with thrombosis in a subject in need thereof.

In another aspect, the present disclosure provides an anti-platelet agent for use in treating thrombosis or a disease or condition resulting from or associated with thrombosis in a subject in need thereof, wherein the anti-platelet agent is for simultaneous, sequential or separate administration with a thrombolytic agent and/or an anti-coagulant.

In another aspect, the present disclosure provides a method of improving the efficacy of a thrombolytic agent administered to a subject in need thereof, the method comprising simultaneously, separately or sequentially administering to the subject an anti-platelet agent.

In another aspect, the present disclosure provides a method of reducing risk of bleeding in a subject receiving a thrombolytic agent and/or an anti-coagulant, the method comprising simultaneously, separately or sequentially administering to the subject an anti-platelet agent.

In another aspect, the present disclosure provides a method of inhibiting re-thrombosis in a subject receiving a thrombolytic agent and/or an anti-coagulant, the method comprising simultaneously, separately or sequentially administering to the subject an anti-platelet agent.

In another aspect, the present disclosure provides a method of inhibiting re-thrombosis in a subject who has received or is considered appropriate to receive a thrombectomy, and/or who has been treated with a stent or is about to be treated with a stent, and/or who is at increased risk of symptomatic intracerebral haemorrhage (sICH), and/or who has been diagnosed with intracranial atherosclerotic disease (ICAD) or as being at risk of developing ICAD, the method comprising administering to the subject an anti-platelet agent, wherein the anti-platelet agent is administered to the subject simultaneously, separately or sequentially with a thrombolytic agent and/or an anticoagulant.

Particular embodiments of each aspect are described throughout the specification, including in the dependent claims.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1 illustrates the in-situ carotid artery thrombolysis (iCAT) stroke model. The iCAT model incorporates thrombotic occlusion of the common carotid artery induced by electrolytic injury to allow for real-time monitoring of occlusion and recanalisation events (A). Following thrombotic occlusion of the carotid artery, transient stenosis of the contralateral carotid artery induces ipsilateral cerebral hypoperfusion sufficient to induce infarction (<25% baseline flow), monitorable with laser doppler flowmetry over the MCA territory (B). Cerebral perfusion analysis with laser speckle contrast imaging at 90 minutes post stroke onset is highly predictive of 24-hour outcome (C), including infarct progression and mortality (D).

FIG. 2 illustrates a model of occlusive thrombus formation induced by electrolytic injury of the mouse carotid artery (herein referred to as the “carotid artery thrombosis model”). Panel A provides schematics illustrating the induction of thrombotic occlusion and administration of test therapeutic agents, as described herein. Panel B shows transverse sections of the mouse carotid artery removed at the completion of experiments, fixed and stained with Carstair's stain to identify platelets (purple), fibrin (crimson red), RBCS (orange/brown) and collagen (blue) rich regions; where the left panel depicts a post sham experiment, the middle and right panels depict post-electrolytic injury 10 minutes (middle) and 60 minutes post-occlusion (right).

FIG. 3 illustrates the occurrence of transient recanalisation and re-thrombosis following rt-PA therapy in a mouse model of thrombotic occlusion. The bar graph represents the percentage of animals presenting with each category of blood flow, where “n” represents the total number of experiments analysed. At the completion of each experiment, vessels were excised, fixed and processed for histology. Sections were stained using a Carstair's stain, with platelets staining blue/purple, fibrin appearing crimson/red, red blood cells staining orange/brown and collagen/vessel wall bright blue (as described). Upper right panel shows carotid artery transection after administration of vehicle only. Lower right panel shows carotid artery transection following rt-PA administration.

FIG. 4 illustrates that anticoagulant therapy improves tPA-mediated recanalisation of the mouse carotid artery. The graph represents the percentage of animals demonstrating each specified category of blood flow (as described in Example 2 herein), where n represents the total “n” animals in each cohort.

FIG. 5 illustrates that adjunctive antiplatelet agents facilitate rtPA-mediated thrombolysis and reduce re-thrombosis. The graph represents the percentage of animals demonstrating each specified category of blood flow, where n represents the total “n” animals in each cohort. Treatment dose regimen: rtPA—tissue plasminogen activator (10 mg/kg); TGX221 (2.5 mg/kg); AZD6482 (2.5 mg/kg).

FIG. 6 illustrates that the PI 3-kinase beta (PI3Kβ) inhibitors TGX221 and AZD6482 are equipotent—achieving comparative anti-platelet efficacy in vivo when combined with rtPA to facilitate thrombolysis. Upper panel shows carotid blood flow over time following administration of rtPA with TGX221. Lower panel shows carotid blood flow over time following administration of rtPA with AZD6482.

FIG. 7 illustrates that co-administration of the antiplatelet TGX221 (equivalent to AZD6482) together with an anticoagulant (argatroban) and thrombolytic agent (rtPA) significantly improves carotid artery recanalisation and prevents re-occlusion. The bar graph represents the percentage of animals presenting with each category of blood flow, where “n” represents the total number of experiments analysed.

FIG. 8 illustrates that TGX221/AZD6482 does not increase tail bleeding—(i) alone; (ii) when combined with a thrombolytic agent (e.g. rt-PA); or (iii) in a triple therapy combination with a thrombolytic agent and an anticoagulant (e.g. argatroban).

FIG. 9 illustrates that Integrilin improves recanalisation in combination with argatroban and rt-PA, however at the expense of an increase in bleeding and mortality.

FIG. 10 illustrates that ‘triple therapy’ with an anti-platelet agent, an anticoagulant and an antithrombotic agent (exemplified here by TGX221-argatroban-tPA) reduced brain infarction and stroke-related mortality, with an excellent functional outcome. Mice were treated intravenously with vehicle, single, dual or triple therapy at 5 minutes after stroke onset and recovered to 24 hours. Outcome was classified according to functional deficit (assessed with travel distance in open-field analysis), cerebral infarction (assessed with TTC staining) and cerebral perfusion at 24-hours. Mild, moderate and severe function was assessed relative to function of sham animals. The graph represents the percentage (%) of animals presenting each score

FIG. 11 illustrates that ‘triple therapy’ with an anti-platelet agent, an anticoagulant and an antithrombotic agent (exemplified here by TGX221-argatroban-tPA) improves carotid recanalisation and cerebral perfusion, and reduces infarct volume 24 hours post recovery. Upper row shows imaging of cerebral perfusion 90 minutes post-stroke onset, carotid recanalisation 60 minutes post occlusion and imaging of infarct volume following vehicle administration. Lower row shows imaging of cerebral perfusion 90 minutes post-stroke onset, carotid recanalisation 60 minutes post occlusion and imaging of infarct volume following administration of TGX221-argatroban-tPA.

FIG. 12 illustrates that ‘triple therapy’ with an anti-platelet agent, an anticoagulant and an antithrombotic agent (exemplified here by TGX221-argatroban-tPA and AZD-argatroban-tPA) improves lysis of aged clots. The bar graph represents the percentage of animals presenting with each category of blood flow, where “n” represents the total number of experiments analysed (values indicated in white at the base of each bar).

### DETAILED DESCRIPTION

### General Techniques and Definitions

Unless specifically defined otherwise, all technical and scientific terms used herein shall be taken to have the same meaning as commonly understood by one of ordinary skill in the art (e.g. immunology, molecular biology, immunohistochemistry, biochemistry, oncology, and pharmacology).

The present disclosure is performed using, unless otherwise indicated, conventional techniques of molecular biology, recombinant DNA technology, immunology and pharmacology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition., 1995), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment or embodiment of the present disclosure may be applied mutatis mutandis to any other aspect or embodiment or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise. For example, a reference to “a bacterium” includes a plurality of such bacteria, and a reference to “an allergen” is a reference to one or more allergens.

Herein the term “about” encompasses a 10% tolerance in any value(s) connected to the term. For the avoidance of doubt, it is to be understood that the term “about” includes a specific reference to the integer (e.g. “about 10” is to be understood as including an explicit reference to 10).

The term “and/or”, e.g., “X and/or Y” shall be understood to mean either “X and Y” or “X or Y” and shall be taken to provide explicit support for both meanings or for either meaning.

Throughout the present specification, various aspects and components of the disclosure can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 2, from 1 to 3, from 1 to 4, from 2 to 3, from 2 to 4, from 2 to 5, from 3 to 4 etc., as well as individual and partial numbers within the recited range, for example, 1, 2, 3, 4, and 5. This applies regardless of the breadth of the disclosed range. Where specific values are required, these will be indicated in the specification.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

### Treatment

As used herein, the terms “treating”, “treat” or “treatment” and variations thereof, refer to clinical intervention designed to alter the natural course of the subject or cell being treated during the course of clinical pathology. Desirable effects of treatment include, for example, decreasing the rate of disease progression, ameliorating or palliating the disease state, remission or improved prognosis. Thus, desirable effects include tissue reperfusion, or restoration of blood flow through an occluded vessel. As used herein, the term “improved” shall be understood to mean decreased mortality, increased magnitude of response, decreased timing of treatment, decreased disease progression, decrease of pathological symptoms for the subject. Accordingly, an improved response to the methods of treatment disclosed herein includes an improvement in one or more effects described herein. For example, the treatments disclosed herein may result in a reduction in any one or more of: thrombus formation, vascular resistance, vascular obstruction, vascular occlusion, number of thrombi, extent of thrombus formation throughout the vasculature, one or more inflammatory mediators, level of serum ferritin, bleeding, difficulty breathing, or other effects disclosed herein or known to result from thrombosis or a disease or condition resulting from or associated with thrombosis. The treatments disclosed herein may result in an increase in any one or more of: thrombus degradation, blood oxygen level, blood platelet level, blood clotting factors, or other effects disclosed herein or known to result from thrombosis or a disease or condition resulting from or associated with thrombosis. The reduction or increase may be any measurable reduction or increase, and may be determined relative to the development or occurrence of that symptom in a subject who does not receive the treatments disclosed herein.

The terms “treat” or “treating” or the like as used herein may be used interchangeably with the terms “inhibit” or “inhibiting”. The term “inhibit” or “inhibiting” shall be taken to mean hinder, reduce, restrain or prevent one or more effects described herein of thrombosis, or a disease or condition resulting from or associated with thrombosis, relative to the development or occurrence of that effect in the absence of any of the treatments disclosed herein.

### Thrombosis

As used herein, the term “thrombosis” includes the formation of a thrombus. The term “thrombus” means a solid mass of platelets and/or fibrin and other components of blood that forms locally in a vessel. Arterial and venous thrombi differ in composition and appearance. Arterial thrombi are typically composed primarily of platelet aggregates, giving the appearance of “white thrombi”, whereas venous thrombi are composed largely of fibrin and red blood cells and are therefore often known as “red thrombi”. Colloquially, a thrombus is referred to as a “blood clot”.

As used herein, the term “treating thrombosis” or “treating a disease or condition resulting from or associated with thrombosis” includes methods of dissolving a thrombus and methods of inhibiting thrombus formation. Thus, the methods disclosed herein may be therapeutic, prophylactic, or both simultaneously.

### Disease Conditions

The methods disclosed herein can be used in the treatment of thrombosis and/or a disease or condition resulting from or associated with thrombosis. The disease or condition resulting from or associated with thrombosis may be characterised at least in part by damage to the endothelial cells of the vasculature. The damage may result from a variety of causes. For example, the damage may result from physical or biochemical insult to the endothelial cells.

The diseases or conditions resulting from or associated with thrombosis include, but are not limited to: stroke (in particular ischemic stroke, such as acute ischemic stroke), myocardial infarction (in particular acute myocardial infarction), angina, transient ischaemic attacks, coronary artery disease, peripheral vascular disease, conditions with a diffuse thrombotic/platelet consumption component, such as disseminated intravascular coagulation (DIC), thrombotic thrombocytopaenic purpura, haemolytic uraemic syndrome, thrombotic complications of septicaemia, acute respiratory distress syndrome (ARDS), anti-phospholipid syndrome, heparin-induced thrombocytopaenia and pre-eclampsia/eclampsia; venous thrombosis such as deep vein thrombosis, pulmonary embolism and venoocclusive disease; haematological conditions, such as myeloproliferative disease, including thrombocythaemia, sickle cell disease, percutaneous coronary interventions (PCI) or interventions in other vessels, stent placement, endarterectomy, coronary and other vascular graft surgery, thrombotic complications of surgical or mechanical damage, such as tissue salvage following accidental or surgical trauma, reconstructive surgery including skin and muscle flaps, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis, in inflammatory conditions such as asthma and chronic obstructive pulmonary disease (COPD), in which platelets and platelet-derived factors are implicated in the immunological disease process, deep vein thrombosis (DVT), pulmonary embolism (PE) and COVID-19 related thrombosis. Thus, the methods disclosed herein may be used to treat a thromboinflammatory disorder, or to treat a subject suffering from or at risk of suffering from acute lung injury. For example, the diseases or conditions resulting from or associated with thrombosis include stroke (in particular ischemic stroke, such as acute ischemic stroke), myocardial infarction (in particular acute myocardial infarction), deep vein thrombosis (DVT), pulmonary embolism (PE) and COVID-19 related thrombosis. The methods disclosed herein are particularly effective in the treatment of stroke (in particular ischemic stroke, such as acute ischemic stroke). The methods disclosed herein are also particularly effective in the treatment of myocardial infarction (in particular acute myocardial infarction). The methods disclosed herein are also effective in the treatment of deep vein thrombosis (DVT), pulmonary embolism (PE) and COVID-19 related thrombosis.

It will be appreciated that the methods disclosed herein can be used to treat a thromboembolism. The embolism may occlude a vessel in another organ or region of the subject's body, such as the lungs, brain, gastrointestinal tract, kidneys, leg, or other organ.

The methods disclosed herein can be used to inhibit re-thrombosis or reocclusion of a blood vessel in a subject already suffering from thrombosis or an occluded blood vessel. The methods disclosed herein can also be applied in order to improve reperfusion of tissue, or to restore blood flow through a vessel in a subject.

The thrombosis may occur locally in any one or more of a subject's arteries, veins, microvasculature or peripheral vasculature.

### Subject

As used herein, the term “subject” refers to any animal, for example, a mammalian animal, including, but not limited to humans, non-human primates, livestock (e.g. sheep, horses, cattle, pigs, donkeys), companion animals (e.g. pets such as dogs and cats), laboratory test animals (e.g. mice, rabbits, rats, guinea pigs), performance animals (e.g. racehorses, camels, greyhounds) or captive wild animals. In one embodiment, the “subject” is a human. Typically, the terms “subject” and “patient” are used interchangeably, particularly in reference to a human subject.

### Anti-Platelet Agent

Any known anti-platelet agent may be used in the methods disclosed herein. In addition, an anti-platelet agent may be identified as such by performing one or more known platelet aggregation assays, wherein a test agent is identified as an anti-platelet agent if it inhibits platelet aggregation. One suitable assay of platelet aggregation, for example, is described in WO2004016607. Alternatively, any available platelet function analyser (PFA) may be used to determine identify an agent as an anti-platelet agent. PFAs are commercially available, including the PFA-100 (Available from Siemens, Munich, Germany).

Known anti-platelet agents that can be used in the methods disclosed herein include, for example, acetylsalicylic acid (Aspirin, Asaphen, Entrophen, Novasen), a COX-1 inhibitor, a P2Y12 receptor antagonist (including, for example, any one or more of clopidogrel (Plavix), ticagrelor (Brilinta), prasugrel (Effient), ticlopidine (Ticlid), Cangrelor), a reversible or an irreversible P2Y12 receptor antagonist, Iloprost, Prostacyclin, a phosphodiesterase 3 (PDE3) inhibitor (including, for example, Dipyridamole), dipyridamole/aspirin (Aggrenox), an NO derivative; a protease activated receptor 1 (PAR1) inhibitor (or a thrombin receptor inhibitor) (including, for example, Vorapaxar), a Glycoprotein IIb/IIIa inhibitor (including, for example, eptifibatide (Integrilin), Aggrastat, Abciximab (ReoPro)), or others. Any anti-platelet agent approved for therapeutic use in humans by any regulatory authority may be used in the methods and uses disclosed herein. Thus, for example, any anti-platelet inhibitor approved by the US FDA for therapeutic use in humans may be used, for example, Aspirin, Yosprala, Abciximab, Eptifibatide, Tirofibran, Ticlopidine, Clopidogrel, Prasugrel, Ticagrelor, Cangrelor, Dipyridamole, Clisostazol or Vorapaxar. Any one or more of the anti-platelet agents disclosed herein may be used in the methods or uses disclosed herein, in any combination.

The anti-platelet agent may be a PI 3-kinase beta inhibitor. Suitable PI 3-kinase beta inhibitors include, but are not limited to, those described in WO2004016607. The PI 3-kinase beta inhibitor disclosed herein may be a compound of Formula (I):

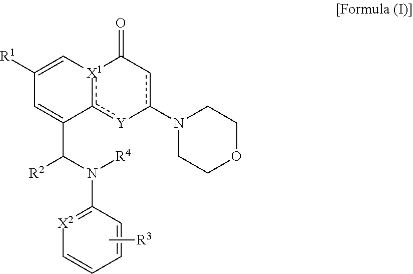

or a pharmaceutically acceptable salt thereof,
wherein,
R¹ is H, OH, OCH₃, OCF₃, F, Cl, CF₃, C₁-C₆ branched or straight chain alkyl, or aryl or (CH₂)ₙ-aryl;
R² is H, C₁-C₆ branched or straight chain alkyl, or aryl or (CH₂)ₙ-aryl in either the R or the S configuration;
R³ is one or more of H, F, Cl, Br, I, CN, CO₂H, CO₂R⁵, NO₂, CF₃, substituted or unsubstituted C₁-C₆ alkyl, substituted or unsubstituted cycloalkyl, substituted or unsubstituted aryl, OCH₃, OCH₂F, OCHF₂, OCF₃, OR⁵, OSO₂-aryl, substituted or unsubstituted amine, NHCOR⁵, NHSO₂R⁵, CONHR⁵, or SO₂NHR⁵;
R₄ is H, C₁-C₆ branched or straight chain alkyl, or aryl or (CH₂)ₙ-aryl;
R⁵ is H, C₁-C₆ branched or straight chain alkyl, or aryl or (CH₂)ₙ-aryl;
n is an integer from 1 to 6;
X¹ is C or N;
X² is C or N; and
Y is N or O.

In some embodiments, the PI 3-kinase beta inhibitor is a compound of Formula (I) or a pharmaceutically acceptable salt thereof, wherein:
R¹ is H, OH, OCH₃, OCF₃, F, Cl, CF₃, C₁-C₆ branched or straight chain alkyl;
R² is H, C₁-C₆ branched or straight chain alkyl, or aryl in either the R or the S configuration;
R³ is one or more of H, F, Cl, Br, CN, CO₂H, CO₂R⁵, NO₂, CF₃, branched or straight chain C₁-C₆ alkyl, substituted or unsubstituted cycloalkyl, substituted or unsubstituted aryl, OCH₃, OCH₂F, OCHF₂, OCF₃, OR⁵, substituted or unsubstituted amine, NHCOR⁵, NHSO₂R⁵, CONHR⁵, or SO₂NHR⁵;

R₄ is H, C₁-C₆ branched or straight chain alkyl, or aryl;
R⁵ is H, C₁-C₆ branched or straight chain alkyl, or aryl;
X¹ is C or N;
X² is C or N; and
Y is N or O.

The phosphoinositide 3-kinase beta inhibitor may be a compound of Formula (II):

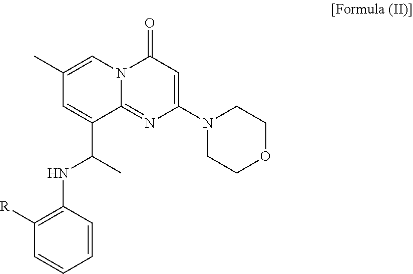

or a pharmaceutically acceptable salt thereof,
wherein R is H or CO₂H.

The phosphoinositide 3-kinase beta inhibitor may be a compound of formula IIa:

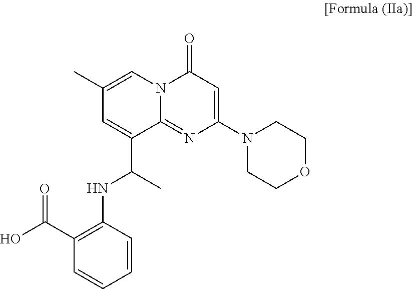 [SMILES: Cc1cc(C(C)Nc2ccccc2C(=O)O)c2nc(N3CCOCC3)cc(=O)n2c1]

or a pharmaceutically acceptable salt thereof.

The phosphoinositide 3-kinase beta inhibitor may be a compound of Formula (III):

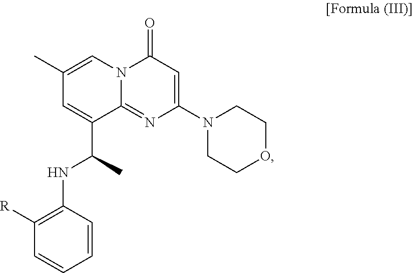

or a pharmaceutically acceptable salt thereof,
wherein R is H or CO₂H. The compound of Formula (III) wherein R is H is also referred to as TGX-221. The compound of Formula (III) wherein R is CO₂H is also referred to as AZD6482.

The phosphoinositide 3-kinase beta inhibitor may be a compound of Formula (IIIa):

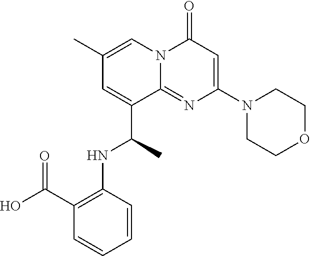 [SMILES: Cc1cc([C@@H](C)Nc2ccccc2C(=O)O)c2nc(N3CCOCC3)cc(=O)n2c1]

or a pharmaceutically acceptable salt thereof.

The PI 3-kinase beta inhibitor may be selected from the group consisting of:
(±)-7-methyl-9-{[methyl(phenyl)amino]methyl}-2-morpholin-4-yl-pyrido[1,2-a]pyrimidin-4-one (TGX-195);
(±)-7-methyl-2-morpholin-4-yl-9-(1-phenylaminoethyl)-pyrido[1,2-a]pyrimidin-4-one (TGX-221);
(±)-7-methyl-2-morpholin-4-yl-9-[1-(4-fluorophenylamino)ethyl]-pyrido[1,2-a]pyrimidin-4-one (TGX-224);
(±)-9-(1-(3,4-difluorophenylamino)ethyl]-7-methyl-2-morpholin-4-yl-pyrido[1,2-a]pyrimidin-4-one (TGX-237);
(±)-9-(1-(2,5-difluorophenylamino)ethyl]-7-methyl-2-morpholin-4-yl-pyrido[1,2-a]pyrimidin-4-one (TGX-238);
(±)-9-(1-(3,5-difluorophenylamino)ethyl]-7-methyl-2-morpholin-4-yl-pyrido[1,2-a]pyrimidin-4-one (TGX-239);
(±)-9-[1-(4-fluoro-2-methylphenylamino)ethyl]-7-methyl-2-morpholin-4-ylpyrido[1,2-a Jpyrimidin-4-one (TGX-240);
(±)-9-[1-(4-chlorophenylamino)ethyl]-7-methyl-2-morpholin-4-yl-pyrido[1,2-a]pyrimidin-4-one (TGX-243);
(±)-9-(1-(3,4-dichlorophenylamino)ethyl]-7-methyl-2-morpholin-4-yl-pyrido[1,2-a]pyrimidin-4-one (TGX-244);
(±)-9-[1-(3fluorophenylamino)ethyl]-7-methyl-2-morpholin-4-yl-pyrido[1,2-a]pyrimidin-4-one (TGX-247);
(±)-9-[1-(3-chlorophenylamino)ethyl]-7-methyl-2-morpholin-4-yl-pyrido[1,2-a]pyrimidin-4-one (TGX-248);
(±)-7-methyl-2-morpholin-4-yl-9-[1-(2-thiazolylamino)ethyl]-pyrido[1,2-a]pyrimidin-4-one (TGX-261);
(±)-7-methyl-9-[1-(3-methylphenylamino)ethyl]-2-morpholin-4-yl-pyrido[1,2-a]pyrimidin-4-one (TGX-262);
(±)-7-methyl-2-morpholin-4-yl-9-[1-(3-trifluoromethylphenylamino)ethyl]pyrido[1,2-a]pyrimidin-4-one (TGX-264);
(±)-7-methyl-2-morpholin-4-yl-9-[1-(2-pyridinylamino)ethyl]-pyrido[1,2-a]pyrimidin-4-one (TGX-295);
(±)-2-({1-[7-methyl-2-(morpholin-4-yl)-4-oxo-pyrido[1,2-a]pyrimidin-9-yl]ethyl}amino)benzoic acid (KN-309);
(±) methyl 2-({1-[7-methyl-2-(morpholin-4-yl)-4-oxo-pyrido[1,2-a]pyrimidin-9-yl]ethyl}amino)benzoate (KN-321);
(±)-2-({1-[7-methyl-2-(morpholi-4-yl)-4-oxo-pyrido[1,2-a]pyrimidin-9-yl]ethyl} amino)benzonitrile (KN-320);
(±)-7-methyl-2-(morpholin-4-yl)-9-(1-{[2-(2H-tetrazol-5-yl)phenyl]amino}ethyl)pyrido[1,2-a]pyrimid-4-one (KN-325); and
(±)-2-(4-morpholinyl)-8[1-(phenylamino)ethyl]-4H-1-benzopyran-4-one (TGX-280).

In some embodiments, the PI 3-kinase beta inhibitor is selected from the group consisting of:
(±)-7-methyl-9-{[methyl(phenyl)amino]methyl}-2-morpholin-4-yl-pyrido[1,2-a]pyrimidin-4-one (TGX-195);
(±)-7-methyl-2-morpholin-4-yl-9-(1-phenylaminoethyl)-pyrido[1,2-a]pyrimidin-4-one (TGX-221);
(±)-9-(1-(3,5-difluorophenylamino)ethyl]-7-methyl-2-morpholin-4-yl-pyrido[1,2-a]pyrimidin-4-one (TGX-239);
(±)-9-[1-(4-chlorophenylamino)ethyl]-7-methyl-2-morpholin-4-yl-pyrido[1,2-a]pyrimidin-4-one (TGX-243);
(±)-9-(1-(3,4-dichlorophenylamino)ethyl]-7-methyl-2-morpholin-4-yl-pyrido[1,2-a]pyrimidin-4-one (TGX-244);
(±)-9-[1-(3-chlorophenylamino)ethyl]-7-methyl-2-morpholin-4-yl-pyrido[1,2-a]pyrimidin-4-one (TGX-248);
(±)-7-methyl-9-[1-(3-methylphenylamino)ethyl]-2-morpholin-4-yl-pyrido[1,2-a]pyrimidin-4-one (TGX-262);
(±)-7-methyl-2-morpholin-4-yl-9-[1-(3-trifluoromethylphenylamino)ethyl]pyrido[1,2-a]pyrimidin-4-one (TGX-264);
(±)-7-methyl-2-morpholin-4-yl-9-[1-(2-pyridinylamino)ethyl]-pyrido[1,2-a]pyrimidin-4-one (TGX-295);
(±)-2-({1-[7-methyl-2-(morpholin-4-yl)-4-oxo-pyrido[1,2-a]pyrimidin-9-yl]ethyl}amino)benzoic acid (KN-309);
(±) methyl 2-({1-[7-methyl-2-(morpholin-4-yl)-4-oxo-pyrido[1,2-a]pyrimidin-9-yl]ethyl}amino)benzoate (KN-321);
(±)-2-({1-[7-methyl-2-(morpholi-4-yl)-4-oxo-pyrido[1,2-a]pyrimidin-9-yl]ethyl} amino)benzonitrile (KN-320);
(±)-7-methyl-2-(morpholin-4-yl)-9-(1-{[2-(2H-tetrazol-5-yl)phenyl]amino}ethyl)pyrido[1,2-a]pyrimid-4-one (KN-325); and
(±)-2-(4-morpholinyl)-8[1-(phenylamino)ethyl]-4H-1-benzopyran-4-one (TGX-280),
   or a pharmaceutically acceptable salt thereof.

The PI 3-kinase beta inhibitor may be selected from the group consisting of:
(±)-7-methyl-2-morpholin-4-yl-9-(1-phenylaminoethyl)-pyrido[1,2-a]pyrimidin-4-one;
(±)-2-({1-[7-methyl-2-(morpholin-4-yl)-4-oxo-pyrido[1,2-a]pyrimidin-9-yl]ethyl} amino)benzoic acid;
(±)-2-({1-[7-methyl-2-(morpholin-4-yl)-4-oxo-pyrido[1,2-a]pyrimidin-9-yl]ethyl} amino)benzonitrile;
(±) methyl 2-({1-[7-methyl-2-(morpholin-4-yl)-4-oxo-pyrido[1,2-a]pyrimidin-9-yl]ethyl} amino)benzoate; and
(±)-7-methyl-2-(morpholin-4-yl)-9-(1-{[2-(2H-tetrazol-5-yl)phenyl]amino)ethyl)pyrido[1,2-a]pyrimid-4-one,
   or a pharmaceutically acceptable salt thereof.

The PI 3-kinase beta inhibitor may be (±)-2-({1-[7-methyl-2-(morpholin-4-yl)-4-oxo-pyrido[1,2-a]pyrimidin-9-yl]ethyl} amino)benzoic acid or a pharmaceutically acceptable salt thereof.

The PI 3-kinase beta inhibitor may be (−)-2-({1-[7-methyl-2-(morpholin-4-yl)-4-oxo-pyrido[1,2-a]pyrimidin-9-yl]ethyl} amino)benzoic acid or a pharmaceutically acceptable salt thereof.

The PI 3-kinase beta inhibitor may be (+)-2-({1S-[7-methyl-2-(morpholin-4-yl)-4-oxo-pyrido[1,2-a]pyrimidin-9-yl]ethyl} amino)benzoic acid or a pharmaceutically acceptable salt thereof.

(−)-2-({1R-[7-methyl-2-(morpholin-4-yl)-4-oxo-pyrido[1,2-a]pyrimidin-9-yl]ethyl}amino)benzoic acid is also referred to as AZD6482. AZD6482 is available from commercial suppliers, such as MedChemExpress (Australia) and Cayman Chemical (United States of America).

The compounds of formula (I), Formula (II) and Formula (IIa) may be prepared by the methods described in WO2004016607. Where these compounds include a chiral centre, the methods and uses described extend to include all enantiomers and diastereoisomers, as well as mixtures thereof in any proportions. The methods and uses described herein further extend to isolated enantiomers (for example, compounds of Formula (III) and Formula (IIIa) or pairs of enantiomers. Methods of separating enantiomers and diastereoisomers are well known to persons skilled in the art. In some embodiments the compounds are racemic mixtures. In other embodiments the compounds are present in enantiomerically pure form. The compounds of formula (III) and Formula (IIIa) may be prepared by the methods described in WO2009093972.

The PI 3-kinase beta inhibitor may be enantiomerically pure (−) 2-[1-(7-methyl-2-(morpholin-4-yl)-4-oxo-4H-pyrido[1,2-a]pyrimidin-9-yl)ethylamino]benzoic acid or pharmaceutically acceptable salts thereof.

As used herein, the term “enantiomerically pure” means (−) 2-[1-(7-methyl-2-(morpholin-4-yl)-4-oxo-4H-pyrido[1,2-a]pyrimidin-9-yl)ethylamino]benzoic acid essentially free from the other enantiomer, i.e. the (+)-enantiomer of 2-[1-(7-methyl-2-(morpholin-4-yl)-4-oxo-4H-pyrido[1,2-a]pyrimidin-9-yl)ethylamino]benzoic acid. Single enantiomers of 2-[1-(7-methyl-2-(morpholin-4-yl)-4-oxo-4H-pyrido[1,2-a]pyrimidin-9-yl)ethylamino]benzoic acid, including the (−) 2-[1-(7-methyl-2-(morpholin-4-yl)-4-oxo-4H-pyrido[1,2-a]pyrimidin-9-y 1)ethylamino]benzoic acid and methods of obtaining the single enantiomers are described in WO2009093972. The term “enantiomerically pure” means e.g. ≥95% enantiomeric excess (ee) of one of the enantiomers of 2-[1-(7-methyl-2-(morpholin-4-yl)-4-oxo-4H-pyrido[1,2-a]pyrimidin-9-yl)ethylamino]benzoic acid. In some embodiments, the pure enantiomers of 2-[1-(7-methyl-2-(morpholin-4-yl)-4-oxo-4H-pyrido[1,2-a]pyrimidin-9-yl)ethylamino]benzoic acid of the present invention may be obtained with high enantiomeric purity, e.g. ≥99.8% enantiomeric excess (ee), e.g. 99.9% ee of (−) 2-[(1R)-(7-methyl-2-(morpholin-4-yl)-25 4-oxo-4H-pyrido[1,2-a]pyrimidin-9-yl)ethylamino]benzoic acid.

The enantiomerically pure (−) 2-[(1R)-1-(7-methyl-2-(morpholin-4-yl)-4-oxo-4H-pyrido[1,2-a]pyrimidin-9-yl)ethylamino]benzoic acid may be in a neutral form. The neutral form may be more stable, easier to handle and store, easier to purify and easier to synthesise in a reproducible manner.

The invention further relates to enantiomerically pure (−) 2-[1-(7-methyl-2-(morpholin-4-yl)-4-oxo-4H-pyrido[1,2-a]pyrimidin-9-yl)ethylamino]benzoic acid, or pharmaceutically acceptable salts thereof, being in a solid state which can be amorphous, at least partly crystalline or substantially crystalline. The crystalline form may be more stable, easier to handle and store, and easier to purify and easier to synthesise in a reproducible manner. Example solid state forms are described in WO2009093972.

In the context of this description, the term “alkyl” refers to straight or branched saturated aliphatic hydrocarbon radical. Preferably, the alkyl group has 1 to 6 carbons as exemplified by methyl, ethyl, propyl, isopropyl, butyl, isobutyl, sec-butyl, t-butyl, pentyl, ipentyl, hexyl and the like. The alkyl group is optionally substituted with one or more groups selected from halogen such as F, Cl, Br or I; CN; CO₂R⁵; NO₂; CF₃; substituted or unsubstituted C₁-C₆ alkyl; substituted or unsubstituted C₃-C₆ cycloalkyl; substituted or unsubstituted aryl; OCF₃, OR⁵, substituted or unsubstituted amine; NHCOR⁵; NHSO₂R⁵; CONHR⁵; or SO₂NHR⁵, wherein R⁵ is H, substituted or unsubstituted C₁-C₆ alkyl, substituted or unsubstituted aryl.

The term “cycloalkyl” refers to non-heterocyclic (i.e., carbocyclic) or heterocyclic ring. Exemplary of non-heterocyclic ring in this regard is substituted or unsubstituted cyclopropane, cyclobutane, cyclopentane, cyclohexane, cyclohexadione, cyclopentanedione, quinone and the like. Suitable heterocycloalkyl groups include substituted or unsubstituted pyrrolidine, piperidine, piperazine, 2-piperidone, azacyclohexan-2-one and morpholine groups. The cycloalkyl group is optionally substituted at one or more positions with halogen such as F, Cl, Br or I; CN; CO₂R⁵; NO₂; CF₃, substituted or unsubstituted C₁-C₆ alkyl; substituted or unsubstituted C₃-C₆ cycloalkyl; substituted or unsubstituted aryl; OCF₃, OR⁵, substituted or unsubstituted amine; NHCOR⁵; NHSO₂R⁵; CONHR⁵; or SO₂NHR⁵, wherein R⁵ is H, substituted or unsubstituted C₁-C₆ alkyl, substituted or unsubstituted aryl.

The term “aryl” refers to an aromatic or heteroaromatic rings. Examples of an aryl group are pyrrolidine, thiophene, pyrrole, pyrazole, imidazole, 1,2,3-triazole, 1,2,4-triazole, oxazole, isoxazole, thiazole, isothiazole, furan, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, tetrazole, benzene, pyridine, pyridazine, pyrimidine, pyrazine, triazine, indene, naphthalene, indole, isoindole, indolizine, benzofuran, benzothiophene, indazole, benzimidazole, benzthiazole, purine, quinolizine, quinoline, isoquinoline, cinnoline, phthalazine, quinazoline, quinoxaline, naphthyridine, pteridine, fluorene, carbazole, carboline, acridine, phenazine, and anthracene. The aryl group is optionally substituted at one or more positions with halogen such as F, Cl, Br or I; CN; CO₂R⁵; NO₂; CF⁵, substituted or unsubstituted C₁-C₆ alkyl; substituted or unsubstituted C₃-C₆ cycloalkyl; substituted or unsubstituted aryl; OCF₃, OR⁵, substituted or unsubstituted amine; NHCOR⁵; NHSO₂R⁵; CONHR⁵; or SO₂NHR⁵, wherein R⁵ is H, substituted or unsubstituted C₁-C₆ alkyl, substituted or unsubstituted aryl.

The term “selective PI 3-kinase β inhibitor” as used herein refers to a compound that inhibits PI 3-kinase β at least >10-fold, preferably >20-fold, more preferably >30-fold more effectively than other isoforms of the PI 3-kinase family. A “selective PI 3-kinase β inhibitor” compound is understood to be more selective for PI 3-kinase β than compounds conventionally and generally designated PI 3-kinase inhibitors such as L Y294002 or wortmannin. Compounds of any type that selectively inhibit PI 3-kinase β expression or activity can be used as selective PI 3-kinase β inhibitors in the methods of the present invention.

The compounds of Formula (I), Formula (II), Formula (IIa), Formula (III) and Formula (IIIa) are also taken to include hydrates and solvates. Solvates are complexes formed by association of molecules of a solvent with the compound.

The compounds may be used in the form of pharmaceutically acceptable salts. Such salts are well known to those skilled in the art. S. M. Berge et al. describe pharmaceutically acceptable salts in detail in J. Pharmaceutical Sciences, 1977, 66:1-19. Pharmaceutically acceptable salts can be prepared in situ during the final isolation and purification of the compounds, or separately by reacting the free base compounds with a suitable organic acid. Suitable pharmaceutically acceptable acid addition salts may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, ascorbic, glucoronic, fumaric, maleic and pyruvic. Suitable pharmaceutically acceptable base addition salts of the compounds include metallic salts made from lithium, sodium, potassium, magnesium, calcium, aluminium, and zinc, and organic salts made from organic bases such as choline, diethanolamine and morpholine.

The compounds of Formula (I), Formula (II), Formula (IIa), Formula (III) and Formula (IIIa) also extend to include all derivatives with physiologically cleavable leaving groups that can be cleaved in viva to provide the compounds.

### Thrombolytic Agent

As used herein, the term “thrombolytic agent” includes an enzyme or compound that is pharmacologically effective in helping to dissolve a thrombus. A thrombolytic agent can be one or a combination of more than one thrombolytic agent. Examples of suitable thrombolytic agents that can be used in the methods and uses described herein include one or more of tissue plasminogen activator, tenectoplase, streptokinase, urokinase, reteplase, prourokinase, anistreplase or other known fibrinolytic enzymes or compounds. However, it will be appreciated by a person skilled in the art that other thrombolytic agents not mentioned above would also be suitable for use in the methods disclosed herein.

For example, the thrombolytic agent can be a tissue plasminogen activator (tPA) or a variant thereof (also known as fibrinokinase; extrinsic plasminogen activator; t-PA; TPA; Activase®). tPA includes recombinant tPA. tPA can be obtained from commercial suppliers, such as Activase®, alteplase available from Genentech, South San Francisco, Calif. Alternatively, tPA can be produced recombinantly or purified from a source.

Another example thrombolytic agent is tenectoplase (also known as TNKase and Metalyse). Tenecteplase is a modified form of human tissue plasminogen activator (tPA) that binds to fibrin and converts plasminogen to plasmin and is produced by recombinant DNA technology.

Another example thrombolytic agent is streptokinase (also known as Streptococcal fibrinolysin; plasminokinase; Awelysin®; Kinalysin®; Kabikinase®; and Streptase), an enzyme elaborated by hemolytic streptococci which hydrolyzes —CONH— links (peptide bonds) and is an activator of plasminogen, thus producing plasmin, which dissolves fibrin. Streptokinase can be produced recombinantly or purified from a source.

Yet another example thrombolytic agent is Urokinase (also known as Abbokinase®; Actosolv®; Breokinase®; Persolv®; Purochin®; Ukidan®; Uronase®; and Winkinase®). Urokinase is serine protease which activates plasminogen to plasmin and is present in mammalian blood and urine. Yet another example thrombolytic agent is Prourokinase (enzyme-activating) (also known as single-chain urokinase-type plasminogen activator; single-chain pro-urokinase; scu-PA; pro-UK; pro u-PA; PUK; Tomieze®; Sandolase®). Prourokinase is a single-chain proenzyme form of urokinase with intrinsic thrombolytic activity and consists of about 411 amino acid residues, with a molecular weight of about 50,000 daltons. Prourokinase can be extracted from urine or from kidney tissue culture and purified or can be produced recombinantly.

Any of the thrombolytic agents disclosed herein may be used in the methods and uses disclosed herein in combination with (i.e., administered simultaneously, sequentially or separately with) an anti-platelet agent (such as a phosphoinositide 3-kinase beta inhibitor, including the inhibitors of Formula (I), Formula (II), Formula (IIa), Formula (III) or Formula (IIIa)) alone. The present disclosure demonstrates that particular advantages can be achieved through the administration of a thrombolytic agent in combination with an anti-platelet agent (and particularly with a phosphoinositide 3-kinase beta inhibitor, including the inhibitors of Formula (I), Formula (II), Formula (IIa), Formula (III) or Formula (IIIa)).

In addition, any of the thrombolytic agents disclosed herein may be used in the methods and uses disclosed herein in combination with (i.e., administered simultaneously, sequentially or separately with) an anti-platelet agent (such as a phosphoinositide 3-kinase beta inhibitor, including the inhibitors of Formula (I), Formula (II), Formula (IIa), Formula (III) or Formula (Ma)) and an anticoagulant (including any of the anticoagulants disclosed herein, such as heparin). The present disclosure also demonstrates that particular advantages can be achieved through the administration of a thrombolytic agent in combination with an anti-platelet agent (and particularly with a phosphoinositide 3-kinase beta inhibitor, including the inhibitors of Formula (I), Formula (II), Formula (IIa), Formula (III) or Formula (IIIa)) and an anticoagulant.

Such advantages include, for example, reduced bleeding (or reduced risk of bleeding) in a subject, or a reduced increase in bleeding (or a reduced increase in risk of bleeding) that often accompanies administration of an anticoagulant and/or a thrombolytic agent. Thus, the combined (i.e., administered simultaneously, sequentially or separately) administration of an anti-platelet agent, a thrombolytic agent and/or an anti-coagulant (and particularly the combined administration of each of an anti-platelet agent, a thrombolytic agent and an anti-coagulant), has been shown herein to be safe for administration to a subject. The advantageous safety profile of the combination therapies disclosed herein represents an improvement over alternative treatment protocols previously used.

### Anti-Coagulant

As used herein, the term “anti-coagulant” includes an enzyme or compound that is pharmacologically effective in preventing or reducing coagulation of blood. Anti-coagulants may also be referred to as blood thinners. The methods and uses described herein encompass the use of one or more anti-coagulants selected from the group consisting of: vitamin K antagonists (or coumarin anticoagulants), low molecular weight heparins (LMWHs), direct thrombin inhibitors (DTIs), or Factor Xa inhibitors. LMWHs include, for example, Bemiparin, Certoparin, Dalteparin, Enoxaparin, Nadroparin, Parnaparin, Reviparin, and Tinzaparin. DTIs include, for example, lepirudin, desirudin, bivalirudin, argatroban, dabigatran, and antithrombin III. Bivalirudin (Angiomax) is available as a powder for injection. Argatroban (Acova) is available for administration by injection. Dabigatran (Pradaxa) is available for administration orally. Antithrombin III (Thrombate III) is available as a powder for injection. Factor Xa inhibitors include, for example, apixaban, fondaparinux, rivaroxaban and edoxaban. Apixaban (Eliquis) is available for administration orally. Fondaparinux (Arixtra) is available for administration by injection. Rivaroxaban (Xarelto) is available for administration orally. Edoxaban (Savaysa) is available for administration orally. The methods and uses described herein also encompass the use of one or more anti-coagulants selected from the group consisting of warfarin, argatroban, hirudin, and heparin.

Any of the above anti-coagulants may be used in the methods and uses disclosed herein. Thus, the anti-coagulant may be any one or more of a vitamin K antagonist, a coumarin anticoagulant, a LMWH, a DTI, or a Factor Xa inhibitor. The anti-coagulant may be any one or more of the specific anticoagulants disclosed herein, in any combination. Any of the anticoagulants disclosed herein may be used in the methods and uses disclosed herein in combination with (i.e., administered simultaneously, sequentially or separately with) an anti-platelet agent (such as a phosphoinositide 3-kinase beta inhibitor, including the inhibitors of Formula (I), Formula (II), Formula (IIa), Formula (III) or Formula (IIIa)) alone. In addition, any of the anticoagulants disclosed herein may be used in the methods and uses disclosed herein in combination with (i.e., administered simultaneously, sequentially or separately with) an anti-platelet agent (such as a phosphoinositide 3-kinase beta inhibitor, including the inhibitors of Formula (I), Formula (II), Formula (IIa), Formula (III) or Formula (IIIa)) and a thrombolytic agent (including any of the thrombolytic agents disclosed herein, such as tPA).

It will be appreciated by persons skilled in the art that additional anticoagulant agents not listed above will also be suitable for use in the methods and uses described herein.

### Administration

The terms “administration of” and/or “administering” compound should be understood to mean providing a compound of the present disclosure to the individual in need of treatment. The compound may be provided by any suitable means. For example, any one or more of the agents or compounds disclosed herein may be administered intravenously or intra-arterially.

The methods of the present disclosure may be performed prior to receiving a thrombolytic agent and/or an anti-coagulant, while the subject is receiving a thrombolytic agent and/or an anti-coagulant, or after the subject has received a thrombolytic agent and/or an anti-coagulant. In one example, the subject is receiving a thrombolytic agent and/or an anti-coagulant. In one example, the subject has been prescribed a thrombolytic agent and/or an anti-coagulant, but is yet to receive the a thrombolytic agent and/or an anti-coagulant. In one example, the subject has received a thrombolytic agent and/or an anti-coagulant, e.g., is undergoing treatment with a thrombolytic agent and/or an anti-coagulant. In one example, the subject is receiving the thrombolytic agent tissue plasminogen activator and an anti-coagulant. The anticoagulant may be, for example, heparin or argatroban. In another example, the subject has received the thrombolytic agent tissue plasminogen activator and is then administered an anti-coagulant in combination with the anti-platelet agent.

The methods of the present disclosure may be performed on a specific patient population. For example, the subject may have undergone or be about to undergo one or more procedures which are suitable for the treatment of stroke (e.g. acute ischaemic stroke). Non limiting examples of such procedures include thrombectomy (also referred to endovascular thrombectomy or EVT), stenting, or any other suitable procedures known in the art.

The methods of the present disclosure may be performed on a subject that has received a thrombectomy (for example, via an arterial catheter). The methods of the present disclosure may be performed on a subject that is considered appropriate to receive a thrombectomy, for example by fulfilling current guidelines including, but not limited to, large vessel occlusion (LVO) in internal carotid artery, proximal Middle Cerebral Artery (MCA) M1 segment, or with tandem occlusion of both cervical carotid and intracranial large arteries presenting within 6 hrs or up to 24 hours after the onset of stroke symptoms. A thrombectomy is a procedure using a clot retrieval device that can be used to remove thrombi that may be resistant to thrombolysis, for example, thrombi in large cerebral vessels. Any suitable clot retrieval device may be used, for example, a stent retriever, or any suitable, commercially available clot retrieval device. The subject may have had poor recanalization or failed recanalization after the thrombectomy. The subject may have experienced cerebral hypoperfusion after the thrombectomy The subject may have experienced re-occlusion after the thrombectomy. The subject may have experienced reduced blood flow after thrombectomy. The subject may have experienced re-thrombosis after the thrombectomy, or the development of one or more additional thrombi after removal of one or more thrombi by thrombectomy.

The methods of the present disclosure may be performed on a subject that has been treated with a stent or is about to be treated with a stent. Any suitable stent may be used, for example, an intracranial stent, a self-expanding intracranial atherosclerotic stent and the like. The subject may have undergone permanent intracranial stenting. The subject may have received more than one stent.

The methods of the present disclosure may be performed on a subject that is at increased risk of symptomatic intracerebral haemorrhage (sICH). Haemorrhagic transformation and its more severe form, parenchymal haemorrhage (PH), is a complication of acute stroke treatments. “sICH” may be defined as a parenchymal haematoma (PH) type I or type II on post-intervention non-contrast CT scan within 36 hours of treatment (modified SITS-MOST definition of sICH) or haemorrhage outside the ischaemic area, associated with a ≥4 point deterioration on the NIHSS (National Institutes of Health stroke scale). Alternative definitions of “sICH” will be understood by a person skilled in the art.

The methods of the present disclosure may be performed on a subject that has been diagnosed with intracranial atherosclerotic disease (ICAD) or who is at risk of developing ICAD. ICAD is highly prevalent in African-American, Asian (China, Japan, South Korea, India), and Hispanic populations. The methods of the present disclosure may be performed on a subject that has been diagnosed with atherosclerotic lesions and/or vessel stenosis within the intracranial vessels.

Any of the combinations of administration of an anti-platelet agent (such as a phosphoinositide 3-kinase beta inhibitor, including the inhibitors of Formula (I), Formula (II), Formula (IIa), Formula (III) or Formula (IIIa) with any of the anticoagulants disclosed herein may be administered to a subject who has received or is considered appropriate to receive a thrombectomy, or who has been treated with a stent or is about to be treated with a stent, or who is at increased risk of symptomatic intracerebral haemorrhage (sICH), or who has been diagnosed with intracranial atherosclerotic disease (ICAD) or as being at risk of developing ICAD.

Similarly, any of the combinations of administration of an anti-platelet agent (such as a phosphoinositide 3-kinase beta inhibitor, including the inhibitors of Formula (I), Formula (II), Formula (IIa), Formula (III) or Formula (IIIa) with any of the thrombolytic agents (such as tPA) disclosed herein may be administered to a subject who has received or is considered appropriate to receive a thrombectomy, or who has been treated with a stent or is about to be treated with a stent, or who is at increased risk of symptomatic intracerebral haemorrhage (sICH), or who has been diagnosed with intracranial atherosclerotic disease (ICAD) or as being at risk of developing ICAD.

Further, any of the combinations of administration of an anti-platelet agent (such as a phosphoinositide 3-kinase beta inhibitor, including the inhibitors of Formula (I), Formula (II), Formula (IIa), Formula (III) or Formula (IIIa) with any of the anticoagulants disclosed herein and any of the thrombolytic agents disclosed herein (such as tPA) may be administered to a subject who has received or is considered appropriate to receive a thrombectomy, or who has been treated with a stent or is about to be treated with a stent, or who is at increased risk of symptomatic intracerebral haemorrhage (sICH), or who has been diagnosed with intracranial atherosclerotic disease (ICAD) or as being at risk of developing ICAD.

The methods and uses disclosed herein involve administration of an agent, such as an antiplatelet agent, at a suitable start point. A suitable start point may be determined by the treating medical team, for example, based on symptoms (including one or more biomarkers) exhibited by the subject. Suitable methods for assessing symptoms are known in the art. For example, the anti-platelet agent may be administered when the subject exhibits any one or more of the following symptoms: an increased vascular resistance; a decreased blood oxygen level; an increased level of a thrombus degradation product; an increased level of one or more inflammatory markers; an increased level of serum ferritin; a decreased level of blood platelets; a decreased level of blood clotting factors; increased bleeding; a requirement for mechanical ventilation. The anti-platelet agent may be started within 12 hours of the subject exhibiting any one or more of said symptoms. For example, the anti-platelet agent may be started within 0.5, 1, 2, 4, 4.5, 5, 6, 8, 10 or 12 hours of the subject exhibiting any one or more of said symptoms. For example, the anti-platelet agent may be started within 2 or 4.5 hours of the subject exhibiting any one or more of said symptoms. For example, the anti-platelet agent may be started within 1, 2, 3, 4.5, 9 or 24 hours in eligible patients. For example, the anti-platelet agent may be started within 1, 2, 3, 4.5, 9 or 24 hours in eligible patients suffering acute stroke and/or acute myocardial infarction. Alternatively, the anti-platelet agent may be administered as soon as possible after the subject exhibits any one or more of the symptoms disclosed herein. The anti-platelet agent may be administered for a duration sufficient to achieve a beneficial therapeutic outcome (e.g., an improvement in any one or more of the symptoms disclosed herein). The anti-platelet agent may be administered for a duration equivalent to the duration of administration of any of the thrombolytic agents or anticoagulants disclosed herein or otherwise administered in the treatment of thrombosis, such as stroke. The anti-platelet agent may be administered for a duration of, for example, about 1 hour, about 2 hours, about 3 hours, about 4 hours, about 5 hours, about 6 hours, about 7 hours, about 8 hours, about 9 hours, about 10 hours, about 12 hours, about 24 hours, about 48 hours, about one week, about one month, about two months, about three months, about four months, about five months or about six months.

In some examples, the methods and uses disclosed herein involve administration of an anti-platelet agent to a subject, wherein the antiplatelet agent is administered to the subject simultaneously, separately or sequentially with a thrombolytic agent and/or an anticoagulant. The anti-platelet agent, thrombolytic agent and/or an anticoagulant may be started within 12 hours of the subject exhibiting any one or more of said symptoms. For example, the anti-platelet agent, thrombolytic agent and/or an anticoagulant may be started within 0.5, 1, 2, 4, 4.5, 5, 6, 8, 10 or 12 hours of the subject exhibiting any one or more of said symptoms. For example, the anti-platelet agent, thrombolytic agent and/or an anticoagulant may be started within 1, 2, 3, 4.5, 9 or 24 hours of the subject exhibiting any one or more of said symptoms. For example, the anti-platelet agent, thrombolytic agent and/or an anticoagulant may be started within 2 or 4.5 hours of the subject exhibiting any one or more of said symptoms.

Administration of an agent may be stopped at a suitable end point. A suitable end point may be determined by the treating medical team, for example based on symptoms (including biomarkers) exhibited by the subject. For example, the anti-platelet agent may be stopped when the subject exhibits any one or more of the following symptoms: decreased vascular resistance; an increased blood oxygen level; a decreased level of a thrombus degradation product; a decreased level of one or more inflammatory markers; a decreased level of serum ferritin; an increased level of blood platelets; an increased level of blood clotting factors; decreased bleeding; no further requirement for mechanical ventilation.

A change in vascular resistance may be a change in pulmonary vascular resistance. Methods of determining vascular resistance are known in the art. One such method is based on the use of echocardiography. Alternatively or in addition, a transcatheter may be used. Invasive or non-invasive methods may be used. A decrease in blood oxygen level may require the subject to be administered oxygen via a mask or a mechanical ventilator or via intubation. Any suitable thrombus degradation product may be monitored. For example, the thrombus degradation product may be a fibrin degradation product such as a D-dimer. Any suitable inflammatory marker may be monitored. For example, the one or more inflammatory markers may comprise C-reactive protein, plasma viscosity and erythrocyte sedimentation rate.

### Dosage

The agents disclosed herein may be administered to a subject in any therapeutically effective amount. As used herein, the term “therapeutically effective amount” includes a non-toxic but sufficient amount of the relevant agent or compound to provide the desired therapeutic effect. It will be appreciated that the desired therapeutic effect may be a desired prophylactic effect. Those skilled in the art will appreciate that the exact amount of an agent or compound required may vary based on a number of factors, though can be determined by a person skilled in the art.

Where the agents disclosed herein are previously known to be suitable for administration at a known dosage (such as an approved dosage), the known dosage may be employed in the methods disclosed herein. It will also be appreciated that, given the improved efficacy of anti-thrombotic agents and/or anticoagulants that has been demonstrated herein to result from co-administration with an anti-platelet agent, the dosage of anti-thrombotic agent and/or anticoagulants used in the methods disclosed herein may be reduced relative to previously used (such as previously approved) dosages.

When the anti-platelet agent is a compound of Formula (III) or Formula (IIIa), the anti-platelet agent may be administered at a dose suitable to maintain a blood plasma concentration of between 0.5 micromolar and 1.5 micromolar, or between 0.8 micromolar and 1.2 micromolar. For example, the anti-platelet agent may be administered at a dose suitable to maintain a blood plasma concentration of about 1 micromolar. A compound of Formula III or Formula (IIIa) may be administered to the subject at a dose of from 30 mg to 185 mg, for example, over a three hour period. For example, the compound of Formula (III) or Formula (IIIa) may be administered to the subject at a dose of 121.5 mg, 30.38 mg, 60.75 mg, or 182.25 mg, for example, over a three hour period.

More generally, a therapeutically effective amount of the PI 3-kinase β inhibitor is expected to be in the range of about 0.05 mg to about 100 mg per kg body weight, or in the range of about 0.05 mg to about 50 mg per kg body weight, or in the range of about 0.05 mg to about 25 mg per kg body weight, or in the range of about 0.5 mg to about 10 mg per kg body weight, or in the range of about 0.5 mg to about 5 mg per kg body weight. One skilled in the art would be able to determine a therapeutically effective amount of a PI 3-kinase β inhibitor, for example, based on the amount required to achieve the desired blood plasma concentration.

The antiplatelet agents used in the methods and uses described herein may be co-administered with a thrombolytic agent, such as t-PA. The thrombolytic agent may be administered in accordance with the product information approved by any regulatory authority. The dosage of t-PA administered to a subject is dependent upon the condition being treated. t-PA may be administered in accordance with the product information approved by any regulatory authority. For example, the product information detailing the approved dosages and indications is publically available online, such as from a regulatory authority (including the FDA) or from a pharmaceutical resource such as MIMS. For example, the recommended dosage for the treatment of acute ischemic stroke in an adult is intravenous (IV) administration at a dose of 0.9 mg/kg (max 90 mg) infused over 60 min with 10% of the total dose administered as an initial IV bolus over 1 min. For pulmonary embolism, the recommended dosage in adults is 100 mg intravenously administered over 2 hours, with heparin therapy initiated or reinstated near the end of or immediately following the t-TPA infusion when the partial thromboplastin time or thrombin time returns to twice normal or less. For acute myocardial infarction, the recommended dosage is based upon patient's weight and should not exceed 100 mg.

When the thrombolytic agent tPA is administered intravenously (optionally with a perfluorochemical emulsion), a therapeutically effective amount of tPA is, in one example, in the range of about 10 to 150 mg administered intravenously over a three hour period. It is preferred that a dose of 100 mg be administered as 60 mg in the first hour (of which 6 to 10 mg is administered as a bolus over 1 to 2 minutes), 20 mg over the second hour and 20 mg over the third hour. For smaller patients (less than 65 Kg), a dose of 1.25 mg/Kg administered over 3 hours may be used. See, e.g., PHYSICIAN'S DESK REFERENCE, Medical Economics Company Inc., Oradell, N.J., 988-989 (1989), the contents of which are herein incorporated by reference.

In the methods described herein, an anti-platelet agent may be administered to a subject that has been administered or is being administered tissue plasminogen activator, in accordance with the product information for t-PA. The tissue plasminogen activator may be administered at a dose of from 0.6 to 0.9 mg/kg intravenously. at a dose of from 0.6 to 0.9 mg/kg intravenously.

In the methods and uses disclosed herein, the thrombolytic agent may be administered at a low dose. Surprisingly, co-administration of an anti-platelet agent with a thrombolytic agent may decrease the dose of thrombolytic agent required to achieve a therapeutic effect, for example, a half or quarter dose of the thrombolytic agent (relative to the dose provided in the product information) may be used. This has the advantage of reducing the risk of bleeding, and especially when administered with anticoagulants or agents that alter platelet function such as aspirin. Thus, for example, the thrombolytic agent may be tPA, which may be administered at a low dose. For example, the dose of tPA may be less than about 0.9 mg/kg, or less than about 0.8 mg/kg, or less than about 0.7 mg/kg, or less than about 0.6 mg/kg, or less than about 0.5 mg/kg, or less than about 0.4 mg/kg, or less than about 0.3 mg/kg, or less than about 0.2 mg/kg, or less than or equal to about 0.1 mg/kg. In other embodiments the dose of tPA may be between about 0.1 mg/kg and about 0.9 mg/kg, or between about 0.1 mg/kg and about 0.8 mg/kg, or between about 0.1 mg/kg and about 0.7 mg/kg, or between about 0.1 mg/kg and about 0.6 mg/kg, or between about 0.1 mg/kg and about 0.5 mg/kg, or between about 0.1 mg/kg and about 0.4 mg/kg, or between about 0.1 mg/kg and about 0.3 mg/kg, or between about 0.1 mg/kg and about 0.2 mg/kg. In one embodiment the dose of tPA is about 0.6 mg/kg.

The antiplatelet agents used in the methods and uses described herein may be co-administered with Streptokinase. The dosage of Streptokinase administered to a subject is dependent upon the condition being treated. Streptokinase may be administered in accordance with the product information approved by any regulatory authority. For example, the product information detailing the approved dosages and indications is publically available online, such as from a regulatory authority (including the FDA) or from a pharmaceutical resource such as MIMS. For example, the recommended dosage for acute MI in an adult is intravenous infusion of a total dose of 1,500,000 units within 60 min. For treatment of pulmonary embolism, DVT, arterial thrombosis or embolism, recommended treatment in adults is intravenous administration preferably within 7 days of a loading dose of 150,000 units infused into a peripheral vein over 30 minutes.

The antiplatelet agents used in the methods and uses described herein may be co-administered with Tenecteplase. The dosage of Tenecteplase administered to a subject is dependent upon the condition being treated. Tenecteplase may be administered in accordance with the product information approved by any regulatory authority. For example, the product information detailing the approved dosages and indications is publically available online, such as from a regulatory authority (including the FDA) or from a pharmaceutical resource such as MIMS. The recommended dosage is based on body weight and the administration is via IV bolus injection over 5-10 seconds. The maximum dose is 10,000 IU (50 mg). Tenecteplase has similar clinical efficacy to alteplase (rt-PA) for thrombolysis after myocardial infarction.

The antiplatelet agents used in the methods and uses described herein may be co-administered with Reteplase. The dosage of Reteplase administered to a subject is dependent upon the condition being treated. Reteplase may be administered in accordance with the product information approved by any regulatory authority. For example, the product information detailing the approved dosages and indications is publically available online, such as from a regulatory authority (including the FDA) or from a pharmaceutical resource such as MIMS. Reteplase is may be administered as a lO+lO U double bolus injection. 10 U of reteplase corresponds to 17.4 mg of reteplase protein mass.

The antiplatelet agents used in the methods and uses described herein may be co-administered with Anistreplase. The dosage of Anistreplase administered to a subject is dependent upon the condition being treated. Anistreplase may be administered in accordance with the product information approved by any regulatory authority. For example, the product information detailing the approved dosages and indications is publically available online, such as from a regulatory authority (including the FDA) or from a pharmaceutical resource such as MIMS. The recommended dosage is 30 units administered intravenously over two to five minutes.

The antiplatelet agents used in the methods and uses described herein may be co-administered with Urokinase. The dosage of Urokinase administered to a subject is dependent upon the condition being treated. Urokinase may be administered in accordance with the product information approved by any regulatory authority. For example, the product information detailing the approved dosages and indications is publically available online, such as from a regulatory authority (including the FDA) or from a pharmaceutical resource such as MIMS. The recommended dosage for pulmonary embolism in an adult is a loading dose of 4,400 IU/kg over 10 minutes, followed by a maintenance dose of 4,400 IU/kg/hr over 12 hours.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### EXAMPLES

The invention disclosed herein will now be further described by reference to the following non-limiting examples.

### Example 1: Occlusive Thrombus Formation Induced by Electrolytic Injury of the Mouse Carotid Artery (Followed by Stroke Induction where Indicated)

An experimental mouse model was prepared as follows.

### Materials

rtPA was purchased from Boehringer Ingelheim Pty Ltd (North Ryde, NSW, Australia), dialysed and activity assessed as described previously (Samson, Nevin & Medcalf. J T H. 2008.6(12):2218-2220). Integrilin (Eptifibatide) was sourced from Schering, whilst argatroban (Argatra) was purchased from Mitsubishi Pharma.

### Animals and Surgical Preparation

C57Bl/6J mice were purchased from Australian BioResources (ABR, NSW, Australia), or bred at the Laboratory Animal Services (LAS) at the University of Sydney, Australia. All animals were housed in a 12 h light/dark cycle with access to food and water ad libitum. For all studies, male mice aged between 8-12 weeks old (20-30 g) were used. All studies were approved by the University of Sydney Animal Ethics Committee (2014/647; 2018/1343; 2018/1331) in accordance with the requirements of the Australian Code of Practice for the Care and Use of Animals for Scientific Purposes (National Health and Medical Research Council (NHMRC) Australian code for the care and use of animals for scientific purposes, 8th edition. Canberra: National Health and Medical Research Council (2013)).

Anaesthesia was induced with 5% Isoflurane and maintained throughout the procedure at 1.5-2.5% (1-1.2 L/min) oxygen via a nose cone. A topical local anaesthetic was applied at incision sites (Xylocaine 1%, AstraZeneca, NSW, Australia). Rectal temperature was maintained at 37° C. with a feedback-controlled thermoblanket (Harvard Apparatus Ltd., Kent, UK). Poly Visc™ Lubricating Eye Ointment was applied prior to procedures (Alcon Laboratories Pty Ltd, NSW, Australia). Mice were positioned in a stereotaxic frame to allow for rotation between the prone position for imaging and supine position for surgical manipulation of the carotid artery (SGM-4 head holder for mice, Narishige Scientific Instrument Laboratory, Japan).

A medial incision was made to the ventral side of the neck to access the left and right carotid arteries, where either one or both carotid arteries were isolated from the vagus nerve and surrounding connective tissue. An ultrasound Doppler flow probe was attached to the left carotid artery to monitor blood flow (0.5 mm i.d, MC0.5PSB-NH-JN-WC60-CRA10-GA, Transonic Systems Inc, NY, USA). The flow probe was connected to a Doppler flow meter (TS420, Transonic Systems Inc., NY, USA) with data recorded using a PowerLab data acquisition system (ADInstruments, NSW, Australia). Mean blood flow was recorded with LabChart software (version 7.0, ADInstruments, NSW, Australia) and corrected for body weight (ml/min/100 g).

### Thrombotic Occlusion of the Carotid Artery

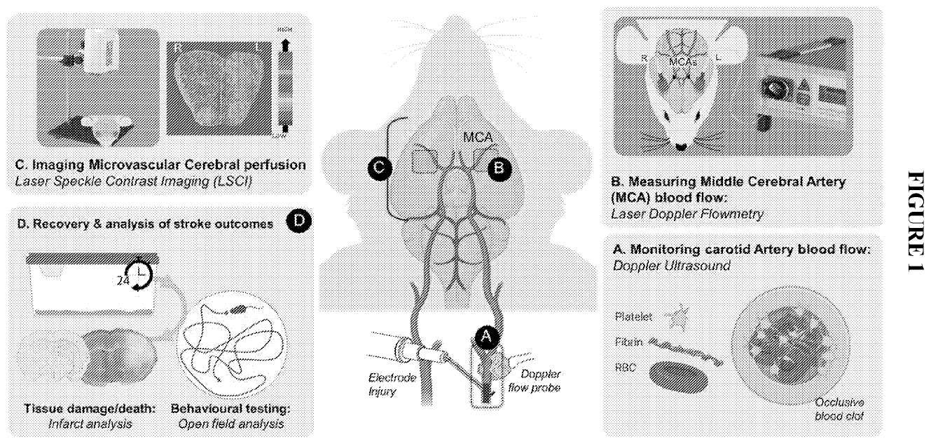

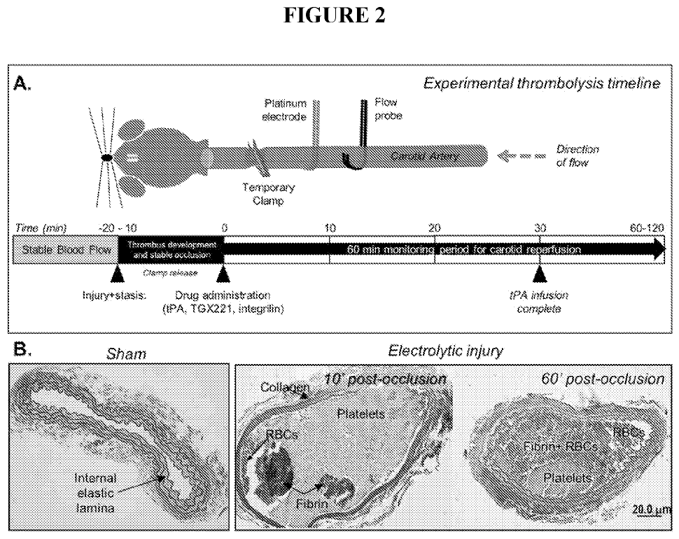

Thrombosis of the left carotid artery was induced with electrolytic injury (carotid artery thrombosis model), as described previously (FIGS. 1 and 2A) (Sturgeon. S. A. J C, Angus. J. A., Wright. C. E. “Adaptation of the folts and electrolytic methods of arterial thrombosis for the study of anti-thrombotic molecules in small animals.” Journal of Pharmacological and Toxicological Methods. 2006; 53:20-29; Mangin P, Yap C L, Nonne C, Sturgeon S A, Goncalves I, Yuan Y, et al. “Thrombin overcomes the thrombosis defect associated with platelet gpvi/fcrgamma deficiency.” Blood. 2006; 107:4346-4353; Schoenwaelder S M, Ono A, Sturgeon S, Chan S M, Mangin P, Maxwell M J, et al. “Identification of a unique co-operative phosphoinositide 3-kinase signaling mechanism regulating integrin alpha iib beta 3 adhesive function in platelets.” J Biol Chem. 2007; 282:28648-28658; Schoenwaelder S M, et al. “14-3-3zeta regulates the mitochondrial respiratory reserve linked to platelet phosphatidylserine exposure and procoagulant function.” Nature Communications. 2016; 7:1-15). To administer the electrolytic injury, a hook-shaped platinum electrode was positioned under the left carotid artery and the carotid artery was clamped distally to induce stasis (Micro Serrefine Clamp, 18055-05, Fine Science Tools, Canada). An electrical current (8 mA) was delivered continuously for 3 minutes through the platinum electrode using a lesion making device (Model 53500, Ugo Basile, Comerio, VA, Italy), and the site was constantly flushed with saline for the duration of injury to enable efficient conduction of current to the vessel. Immediately after injury the clamp was released, the flow probe replaced, and blood flow monitored for occlusion (indicated by reduction of blood flow to 0 ml/min/100 g). Vascular injury induced by electrical injury was assessed using Carstair's stain (FIG. 2B), leading to development of fibrin (red) and platelet (purple)-rich thrombi.

### Delivery of Thrombolytic and Adjunctive Therapies

Thrombolytic and adjunctive therapies were delivered intravenously via the jugular vein at 15-20 minutes following stable carotid artery occlusion. Mice were randomly allocated to treatment, with the operator blinded to treatment allocation. Treatments include single or combination regimens of rtPA (10 mg/kg; 1 mg/kg bolus+9 mg/kg infusion over 30 minutes), hirudin (single bolus 0.3 mg/kg), Integrilin (4 mg/kg repeat bolus 15-minute intervals) or argatroban (80 μg/kg bolus; 40 μg/kg/min infusion). All treatments were delivered simultaneously with rtPA therapy. Treatment was delivered using a Harvard apparatus pump (Cat #704504; Pump Elite 11 I/W Single Syringe Pump, NSW, Australia). Carotid artery blood flow and LDF were monitored concurrently for 60 minutes following treatment onset.

### Additional Surgical Procedures for Ischaemic Stroke Induction

### Transient Stenosis of the Contralateral Carotid Artery for Stroke Induction

Following ten minutes of carotid artery occlusion (defined as flow of 0 ml·min·100 g), ligature-induced stenosis of the right (contralateral) carotid artery was commenced to reduce ipsilateral perfusion <25% of baseline, whilst maintaining contralateral perfusion >25% of baseline for the duration of ischaemia (25, 45, 60 minutes). Perfusion was continuously monitored with laser doppler flowmetry (LDF). Following ischaemia, the ligature was removed to restore maximal blood flow through the contralateral carotid artery and animals were recovered for 24 hours post occlusion and assessed for cerebral infarction and functional outcomes.

### Cerebral Perfusion Monitoring

Cerebral perfusion monitoring was conducted with laser speckle contrast imaging (LSCI) and laser doppler flowmetry (LDF) through the mouse skull. A longitudinal incision was made along the scalp, above the midline and over bregma, with a crosswise incision made between the ears to allow the skin to be retracted.

(i) Laser Speckle Contrast Imaging (LSCI): Laser Speckle Contrast Imaging (LSCI) of brain perfusion (designated flux units) was obtained using a moorFLPI-2 blood flow imager and associated software (Moor FLPI-2 Measurement V1.1, Moor Instruments, UK). Imaging was performed prior to manipulations of the carotid arteries (3 minutes, “baseline”), from 15-30 minutes after contralateral stenosis (“post-bilateral occlusion/stenosis”), and at 24 hours post-occlusion (3 minutes, “24 hour”). Images were captured at 30 second intervals for each recording period, with a temporal filter of 250 frames (1 frame/10 s), and 20 ms exposure. Gain was adjusted for the “Baseline” reading per mouse and used for each subsequent reading (Gain range: minimum 145, maximum 180). Analysis was performed with Moor FLPI-2 Review V4.0 software (Moor Instruments, UK). Hemispheric regions of interest (ROI) were created from the generated colour image of the skull by tracing along the sagittal suture between the eye sockets to lambda, along the lambdoid suture, and the temporalis muscle border. Post-recanalisation and 24-hour flux values were expressed as a percentage of the mean baseline flux for each hemisphere.

ii) Laser doppler flowmetry (LDF): For perfusion monitoring during surgical procedures, LDF was used as previously described with modifications (Tomkins A J et al. Platelet rich clots are resistant to lysis by thrombolytic therapy in a rat model of embolic stroke. Experimental and Translational Stroke Medicine. 2015; 7:1-9). Following baseline LSCI measurements, two fibre-optic laser Doppler probes (P10d and VP10M200ST, Moor Instruments, UK) in custom-made 2×2×2 mm silicone probe holders were affixed to the skull surface. The probes were positioned in each hemisphere over the middle cerebral artery (MCA) territory: 1 mm posterior to bregma and 1 mm lateral to the temporalis muscle border. Perfusion was analysed using moorVMS-LDF2 (Moor Instruments, UK), connected to PowerLab and Labchart software, allowing direct comparison with iCAT carotid blood flow data. “Baseline” was calculated as the mean perfusion of a 5-minute period prior to injury and all subsequent recordings were expressed as a percentage of baseline. Continuous LDF measurements were acquired during the procedure until 10 minutes after the right carotid clamp or stenosis was removed. The probe holders were then removed for post-bilateral occlusion/stenosis LSCI.

### Post-Operative Recovery

Mice were recovered for 24-hour post-occlusion to allow for assessment of cerebral infarction (TTC staining). Mice were recovered post-surgery in a warmed environment (26-28° C.) from 2-24 h post recovery, wherein a warming cabinet (Cat #ASSWC24; Able Scientific, NSW, Australia) was utilised to control the post-operative temperature during overnight recovery.

### Assessment of Cerebral Infarction

For the quantification of stroke volume, mice were deeply anaesthetised with isoflurane and transcardially perfused with cold saline. The brain was removed and sliced into 2 mm sequential coronal brain sections using a Kopf Mouse brain block. Brain slices were incubated at 37° C. in 1% 2,3,5-triphenyl-tetrazolium chloride (TTC, Sigma Aldrich, MO, USA) in saline for 14 minutes (7 minutes each side). After overnight fixation in 10% neutral buffered formalin, brain sections were imaged on a flatbed scanner (Epson Perfection V700) and infarct analysis conducted with Image J software. Of the sections cut, the first four sections were quantified to assess cerebral infarction. An investigator blinded to the experimental groups conducted quantification of infarct volume. Total area and infarct area were calculated for the top and bottom of each section, and then averaged to provide total volume and lesion volume for each section. Infarct volume is presented as the summation of lesion volume for the whole brain.

### Example 2: Transient Recanalisation and Re-Thrombosis Following rt-PA Therapy

C57BL/6 male mice were surgically prepared for electrolytic injury and intravenous treatment administration. Occlusive thrombus formation was induced as described in Example 1 and FIG. 2. 15 minutes following carotid artery occlusion, rtPA (10 mg/kg) was delivered as described in Example 1, or vehicle alone (saline/hepes). Blood flow was monitored for 60 minutes following treatment onset, and recanalisation classified as indicated in Example 1. Briefly, recanalisation was identified by a return of blood flow, and categorised as either stable recanalisation (steady flow), unstable recanalisation (fluctuating flow), transient recanalisation with reocclusion, or no recanalisation.

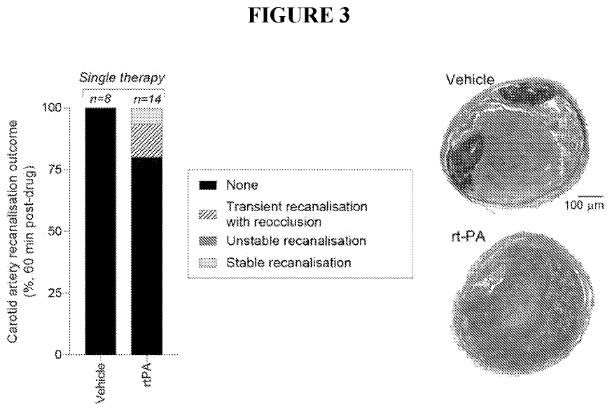

Results are illustrated in FIG. 3. The bar graph represents the percentage of animals presenting with each category of blood flow, where “n” represents the total number of experiments analysed. At the completion of each experiment, vessels were excised, fixed and processed for histology. Sections were stained using a Carstair's stain, with platelets staining blue/purple, fibrin appearing crimson/red, red blood cells staining orange/brown and collagen.vessel wall bright blue (as described).

This data demonstrates that rtPA can induce clot lysis and recanalisation of the carotid in approximately 25% of vessels, however this recanalisation is transient and the majority of vessels reocclude post treatment. Thus, this data demonstrates that rt-PA monotherapy induces transient recanalisation in mouse carotid arteries, and that re-thrombosis post-fibrinolysis is associated with the development of platelet-rich thrombi.

### Example 3: Anticoagulant Therapy Improves tPA-Mediated Recanalisation of the Mouse Carotid Artery

C57BL/6 male mice underwent the carotid artery thrombosis model procedure as described in Example 1, with occlusive thrombus formation induced in the carotid artery with standard injury parameters (8 mA, 3 min). Treatments administered included vehicle alone (saline/hepes), or dual-therapy (rtPA plus hirudin; rtPA plus argatroban), delivered intravenously 5 minutes after onset of stroke induction (corresponding to 15 minutes after occlusion of the carotid artery). rtPA was administered at 10 mg/kg (bolus/infusion regimen), Hirudin (0.3 mg/kg) was given as a single bolus, while argatroban was given as a bolus infusion (80 ug/kg bolus; 40 ug/kg/min infusion 60′).

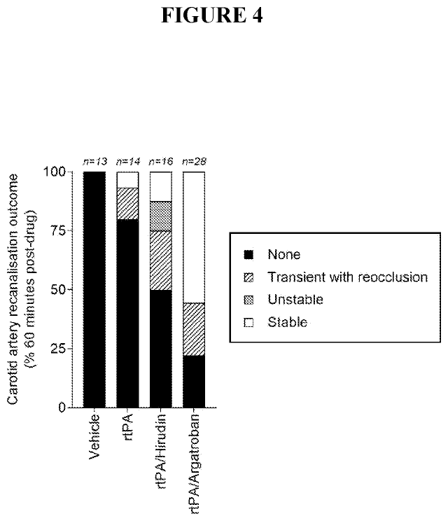

Results are illustrated in FIG. 4. The bar graph represents the percentage of animals demonstrating each specified category of blood flow (as described in Example 2), where n represents the total “n” animals in each cohort.

This data demonstrates that the combination of an anticoagulant (exemplified here using hirudin or argatroban) together with rt-PA improves thrombolysis and reduces vessel reocclusion. Without wishing to be bound by theory, this may result from the ability of hirudin and argatroban to inhibit thrombin released from lysed clots. Notably, the thrombi developing post-rt-PA therapy were platelet-rich.

### Example 4: Adjunctive Antiplatelet Agents Facilitate rtPA-Mediated Thrombolysis and Reduce Re-Thrombosis

Electrolytic injury was induced in the carotid artery of C57BL/6 male mice and thrombolytic therapy administered 15 minutes after occlusion of the carotid artery, as described in Example 1. Treatments administered included vehicle alone (saline/hepes), dual-therapy (rtPA plus TGX221; rtPA plus AZD6482). The treatment dose regimen was as follows: rtPA—recombinant tissue plasminogen activator (10 mg/kg); TGX221 (2.5 mg/kg); AZD6482 (2.5 mg/kg). Recanalisation was monitored for 60 minutes following treatment onset, and occlusion characterised as described in Example 2.

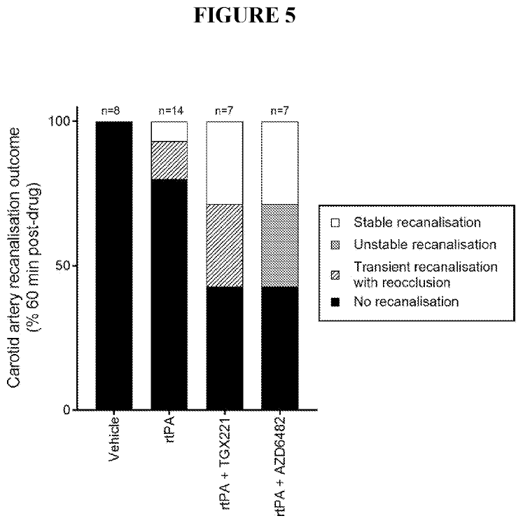

Results are illustrated in FIG. 5. The graph represents the percentage of animals demonstrating each specified category of blood flow, where n represents the total “n” animals in each cohort. This data demonstrates that co-administration of an antiplatelet agent (exemplified here using each of the two PI3Kβ inhibitors—TGX221 and AZD6482) together with rtPA improves thrombolysis and vessel reocclusion over and above that achieved with rtPA alone.

### Example 5: The PI3K Beta Inhibitors TGX221 and AZD6482 are Equipotent—Achieving Comparative Anti-Platelet Efficacy In Vivo when Combined with rtPA to Facilitate Thrombolysis

Electrolytic injury was induced in the carotid artery of C57BL/6 male mice and thrombolytic therapy administered 15 minutes after occlusion of the carotid artery, as described in Example 1. Treatments administered included a comparison of rtPA plus TGX221 versus rtPA plus AZD6482. Recanalisation was monitored for 60 minutes following treatment onset, and occlusion characterised as described in Example 2. Blood flow traces were taken from one representive of 7 independent experiments. The treatment dose regimen was as follows: rtPA—recombinant tissue plasminogen activator (10 mg/kg); TGX221 (2.5 mg/kg); AZD6482 (2.5 mg/kg).

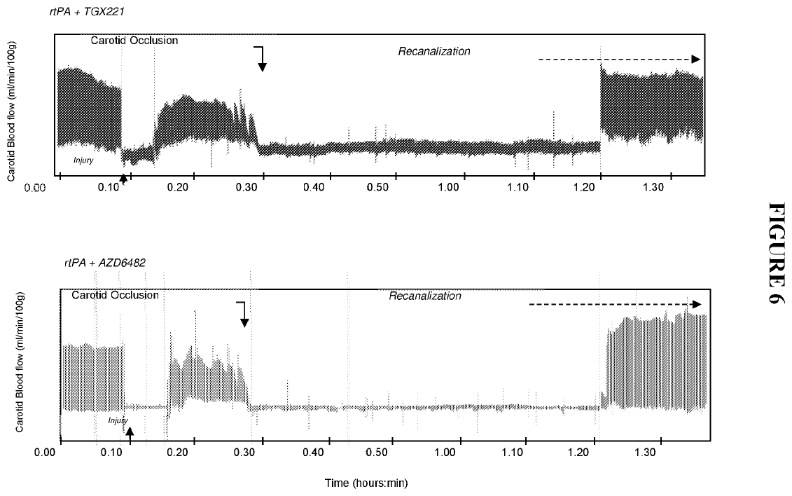

Results are illustrated in FIG. 6. This data demonstrates that the PI3K beta inhibitors TGX221 and AZD6482 are equipotent antiplatelet agents, and achieve similar efficacy in vivo in combination with rtPA both effectively enhancing recanalisation.

### Example 6: Co-Administration of the Antiplatelet TGX221 (Equivalent to AZD6482) Together with an Anticoagulant (Argatroban) and Thrombolytic Agent (rtPA) Significantly Improves Carotid Artery Recanalisation and Prevents Re-Occlusion

C57BL/6 male mice were surgically prepared for electrolytic injury and intravenous treatment administration as described in Example 1. Occlusive thrombus formation was induced in the carotid artery of a C57BL/6 mouse with standard electrolytic injury parameters (8 mA, 3 min). Single, dual or triple therapy was delivered intravenously 15 minutes after occlusion of the carotid artery, where occlusion was characterised as no blood flow through the carotid artery (0 ml·min). Blood flow was monitored for 60 minutes following treatment onset, and recanalisation classified as outlined in Example 2. The treatment dosing regimens were as follows: rtPA—recombinant tissue plasminogen activator (10 mg/kg); Arg—argatroban (80 ug/kg bolus; 40 ug/kg/min infusion 60′); ^{#}Integ-Integrilin (4 mg/kg, bolus every 15 minutes); ^{a}TGX221 (2.5 mg/kg; single bolus 15 minutes prior to lop).

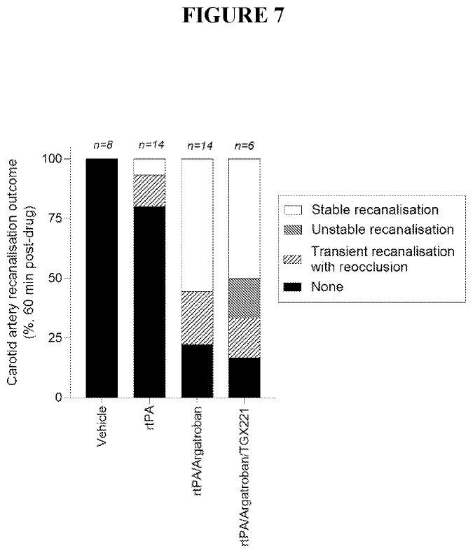

Results are illustrated in FIG. 7. The bar graph represents the percentage of animals presenting with each category of blood flow, where “n” represents the total number of experiments analysed. These studies demonstrate for the first time the significant benefit of co-administration of triple therapy (an anticoagulant plus an anti-platelet agent (exemplified here using TGX221) together with a thrombolytic agent (exemplified here using rtPA) for improving recanalisation as well as reducing reocclusion post thrombolytic agent-induced thrombolysis (e.g., rtPA-mediated thrombolysis).

### Example 7: TGX221 does not Increase Tail Bleeding—Alone, when Combined with a Thrombolytic Agent (e.g. rt-PA) or in a Triple Therapy Combination with a Thrombolytic Agent and an Anticoagulant (e.g. Argatroban)

Bleeding was assessed using a 3 mm tail lop assay. Drug treatments (bolus) were administered 10 min before the start of tail bleeding. When a 30 min infusion was used, the tail bleeding experiment began as soon as the infusion finished. Following drug administration, a 3 mm long section of the tail tip was removed using a scalpel blade and the tail tip immersed in warmed saline. Bleeding was monitored until cessation and the time recorded. The tail was monitored for another 2 min to ensure it did not re-bleed. If the tail did re-bleed, length of time of rebleeding was also recorded. The experiment was ended when bleeding had ceased for at least 2 min, or at 30 min post-tail tip removal. Haemoglobin was quantified using 2 methods [(i, ii) using Abs 575 nm; (iii) using a colorimetric haemoglobin assay, as per the manufacturer's instructions (Sigma Aldrich, Haemoglobin Assay Kit MAK115)]. The treatment dosing regimens were as follows: rtPA (10 mg/kg: 1 mg/kg bolus, 9 mg/kg infusion over 30 min); Integrilin (1-10 mg/kg, bolus every 15 minutes); TGX221 (2.5 mg/kg; single bolus 15 minutes prior to lop); Arg—argatroban (80 ug/kg bolus; 40 ug/kg/min infusion 60). In addition: (i) Dose-response studies of Integrilin were initially performed to characterise the effect of this inhibitor in mouse blood/platelets. Administration of 3.0 and 4.0 mg/kg Integrilin™ resulted in approximately 60% (‘Stroke’ dose) and 80% (‘AMI’ dose) inhibition of ADP-induced aggregation in PPACK-anticoagulated PRP, respectively, and were thus utilised in further experiments to examine efficacy alone or in combination with rtPA. Data are presented as mean±SEM.

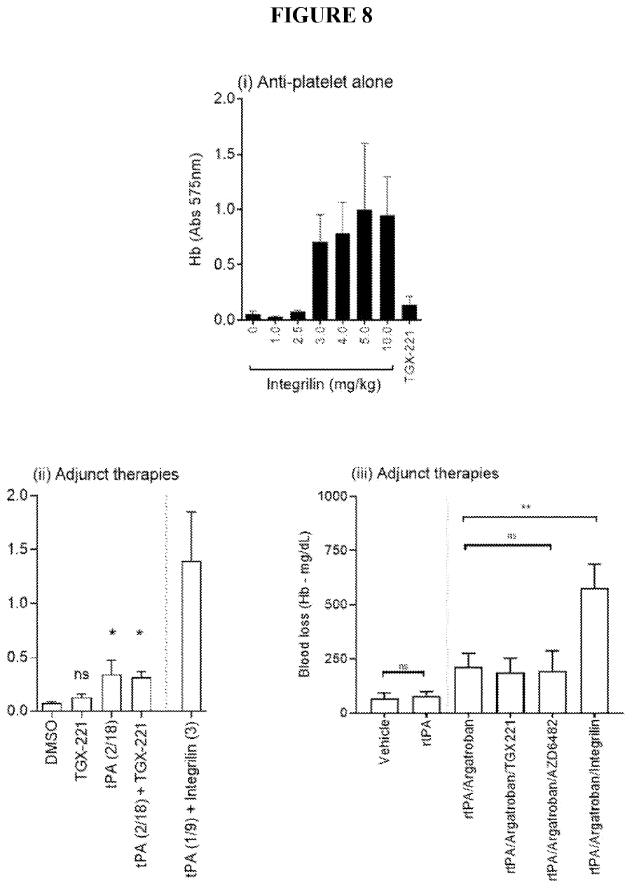

Results are illustrated in FIG. 8. These studies demonstrate the promising bleeding profile when TGX221 (equivalent to AZD6482) is administered with a thrombolytic agent (exemplified here using rt-PA), even when using very high doses of rt-PA—20 mg/kg. This contrasts with Integrilin, which caused a significant increase in bleeding when administered alone (3 mg/kg and above) or in combination with rt-PA (10 mg/kg). Moreover, this data highlights the impressive safety profile of TGX221 when used in a triple therapy combination with a thrombolytic agent (exemplified here using rt-PA) and an anticoagulant (exemplified here using argatroban).

### Example 8: Integrilin Improves Recanalisation in Combination with Argatroban and rt-PA, However at the Expense of an Increase in Bleeding and Mortality

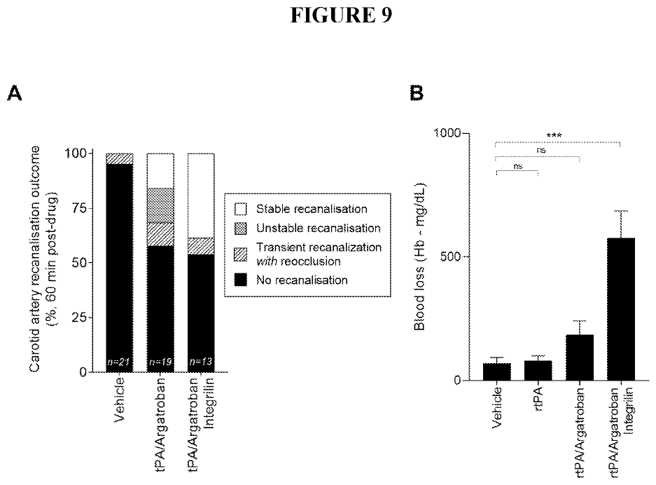

C57BL/6 mice underwent the iCAT procedure for stroke induction. Briefly, electrolytic injury was induced in the carotid artery of C57BL/6 mice and thrombolytic therapy administered 15 minutes after occlusion, as described in Example 1. Recanalisation was monitored for 60 minutes following treatment onset, and occlusion characterised as described in Example 2. Animals were recovered to 24 hours. The treatment dose regimen was as follows: rtPA—tissue plasminogen activator (10 mg/kg); argatroban (80 ug/kg bolus; 40 ug/kg/min infusion 60′); Integrilin (4 mg/kg bolus every 15 minutes). Results are illustrated in FIG. 9A. The graph represents the percentage of animals demonstrating each specified category of blood flow, where n represents the total “n” animals in each cohort. Mortality rate post-surgical recovery was quantified as a percentage of the cohort, and was significantly increased in the rtPA/argatroban/integrilin cohort (>50%), when compare with either vehicle alone or rtPA/Argatoroban cohorts (below 30%).

In addition, 15 minutes following commencement of treatment administration, tail transection (3 mm) was performed, and the tail tip immersed in warm (37° C.) saline to allow for blood collection from the tail (as described under “Methods”). Flow of blood was assessed over 30 minutes and any periods of blood flow cessation and rebleeding noted. Blood monitoring and collection was ceased at 30 minutes after lop, for all mice. Haemoglobin content was assessed in the collected sample using a colorimetric haemoglobin assay, as per the manufacturer's instructions (Sigma Aldrich, Haemoglobin Assay Kit MAK115). Data was analysed using a one-way ANOVA relative to Vehicle. Results are illustrated in FIG. 9B. The bar graph depicts mean±SD, where ***P<0.0005, ^{ns}P>0.05.

These studies demonstrate that the combination of the anti-platelet agent integrilin with the thrombolytic agent rtPA and the anticoagulant argatroban enhances stable recanalisation of a carotid artery occluded by a thrombus. However, the thrombolytic benefit of combining integrilin with rtPA and argatroban was accompanied by an increase in bleeding. These results demonstrate a further advantage of combining the particular anti-platelet agents TGX221 or AZD6482 with a thrombolytic agent such as rtPA and an anti-coagulant such as argatroban, as a safe option for facilitating thrombolysis.

### Example 9: ‘Triple Therapy’ with rtPA, Argatroban and TGX221 Reduced Brain Infarction and Stroke-Related Mortality, with an Excellent Functional Outcome

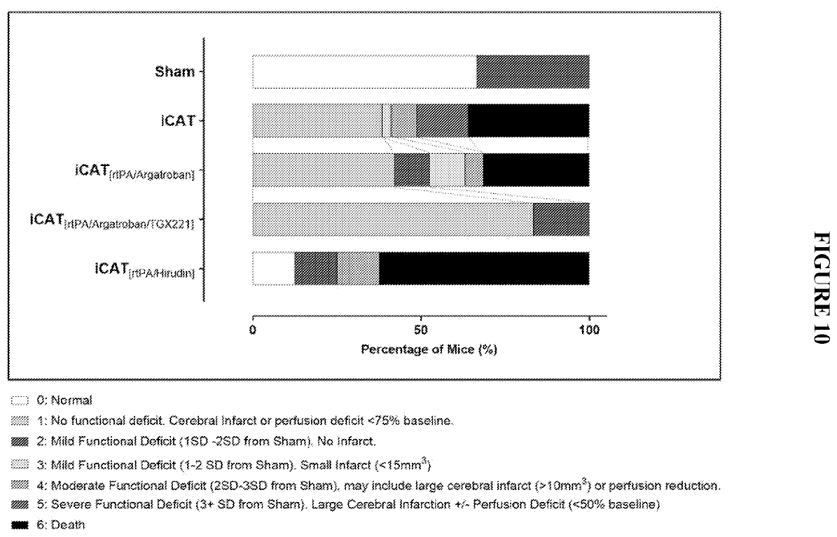

C57BL/6 mice underwent sham or carotid artery thrombosis model procedure, along with additional surgical procedures for ischaemic stroke induction, as described in Example 1. Mice were treated intravenously with vehicle, single, dual or triple therapy at 5 minutes after stroke onset and recovered to 24 hours. Outcome was classified according to functional deficit (assessed with travel distance in open-field analysis), cerebral infarction (assessed with TTC staining) and cerebral perfusion at 24-hours. Mild, moderate and severe function was assessed relative to function of sham animals. The treatment dosing regimens were as follows: rtPA—recombinant tissue plasminogen activator (10 mg/kg); argatroban (80 ug/kg bolus; 40 ug/kg/min infusion 60′); TGX221 (2.5 mg/kg; single bolus). Results are shown in FIG. 10. The graph represents the percentage (%) of animals presenting each score. Total n: No treatment=19; rtPA/argatroban=19; rtPA/argatroban/TGX221=6; rtPA/hirudin=8.

These findings highlight that treatment with a combination of an anti-platelet agent (exemplified here by TGX221), an anticoagulant (exemplified here by argatroban) and an antithrombotic agent (exemplified here by tPA) leads to a high rate of vessel recanalisation, improved cerebral perfusion, reduced infarct burden and improved functional recovery (see Example 10 for examples). Importantly, this triple combination therapy with an anti-platelet agent, an anticoagulant and an antithrombotic agent (exemplified here by TGX221-argatroban-tPA therapy) did not induce bleeding complications and greatly reduced stroke-related mortality at 24 hours. The overall improvements conferred by this triple combination therapy with an anti-platelet agent, an anticoagulant and an antithrombotic agent (exemplified here by TGX221-argatroban-tPA therapy) are highlighted by mild outcomes in the majority of TGX221-argatroban-tPA treated animals when assessed with a modified Rankin Score (mRS) for rodents.

### Example 10: Triple Therapy with rtPA, Argatroban and TGX221 Improves Carotid Recanalisation and Cerebral Perfusion, and Reduces Infarct Volume 24-Hours Post Recovery

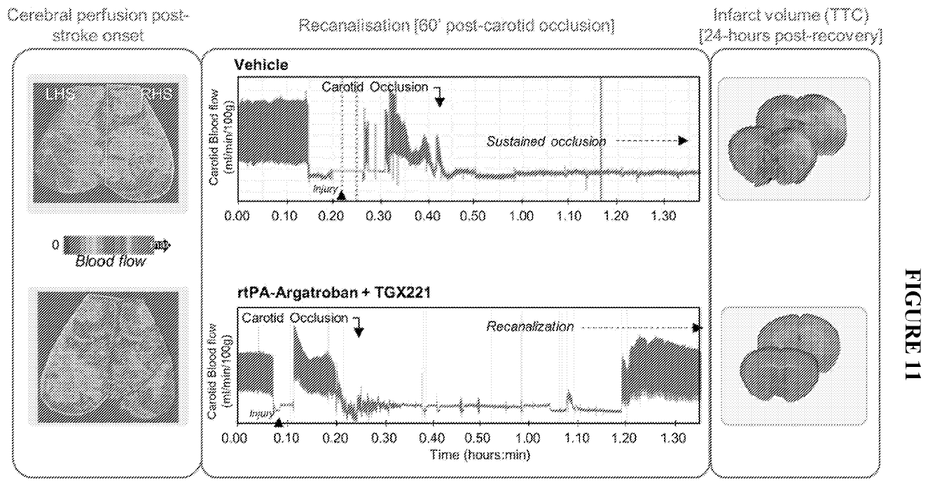

C57BL/6 male mice underwent the carotid artery thrombosis model procedure, along with additional surgical procedures for ischaemic stroke induction, as described in Example 1. This was followed by intravenous treatment administration at the following dosing regimens: rtPA—recombinant tissue plasminogen activator (10 mg/kg); Arg—argatroban (80 μg/kg bolus; 40 μg/kg/min infusion 60′); TGX221 (2.5 mg/kg; single bolus). Specific outcomes are illustrated in FIG. 11.

The images in FIG. 11 represent stroke outcomes from a representative experiment for vehicle and triple therapy treatment (as described in Example 9), demonstrating the improved stroke outcomes following triple therapy, including improved cerebral reperfusion (LEFT), improved blood flow (recanalisation) following carotid occlusion and reduced cerebral infarct post recovery. This data provides further evidence demonstrating the improved therapeutic outcomes resulting from treatment with a combination of an anti-platelet agent (exemplified here by TGX221), an anticoagulant (exemplified here by argatroban) and an antithrombotic agent (exemplified here by tPA).

### Example 11: Triple Therapy with rtPA, Argatroban and TGX221 Improves Lysis of Aged Clots

C57BL/6 male mice were surgically prepared for electrolytic injury and intravenous treatment administration as described in Example 1. Occlusive thrombus formation was induced in the carotid artery of a C57BL/6 mouse with standard electrolytic injury parameters (8 mA, 3 min). Single, dual or triple therapy was delivered intravenously at 15, 120 or 270 minutes after occlusion of the carotid artery, where occlusion was characterised as no blood flow through the carotid artery (0 ml·min). Blood flow was monitored for 60 minutes following treatment onset, and recanalisation classified as outlined in Example 2. The treatment dosing regimens were as follows: rtPA—recombinant tissue plasminogen activator (10 mg/kg); Arg—argatroban (80 ug/kg bolus; 40 ug/kg/min infusion 60; *80 ug/kg bolus; 80 ug/kg/min infusion 60′); TGX221 (2.5 mg/kg; single bolus 15 minutes; \hat{ }TGX221/\hat{ }AZD (2.5 mg/kg; 2× bolus—15 minutes apart).

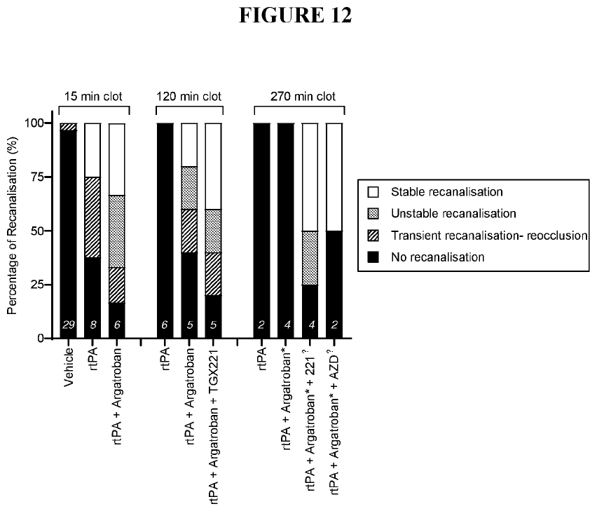

The results are illustrated in FIG. 12. The bar graph represents the percentage of animals presenting with each category of blood flow, where “n” represents the total number of experiments analysed. These studies demonstrate for the first time the significant benefit of co-administration of the triple therapy combination of rtPA, anticoagulant (argatroban) plus an antiplatelet agent (TGX221 or AZD6482) for improving recanalization of older clots that have developed resistance to rtPA-mediated lysis. They also demonstrate the benefit that the triple therapy combination of rtPA, anticoagulant (argatroban) plus an antiplatelet agent (TGX221 or AZD6482) provides in 4.5 hours aged clots that are unresponsive to administration of rtPA/argatroban dual-therapy lysis.

1. A method of treating thrombosis or a disease or condition resulting from or associated with thrombosis, in a subject in need thereof, the method comprising administering to the subject an anti-platelet agent, and wherein the method further comprises the simultaneous, sequential or separate administration of a thrombolytic agent and/or an anti-coagulant.

2. The method of claim 1, wherein the method comprises the simultaneous, sequential or separate administration of a thrombolytic agent and an anti-coagulant.

3. The method of claim 1 or claim 2, wherein the anti-platelet agent is a phosphoinositide 3-kinase beta inhibitor.

4. The method of claim 3, wherein the phosphoinositide 3-kinase beta inhibitor is a compound of formula II or a pharmaceutically acceptable salt thereof: 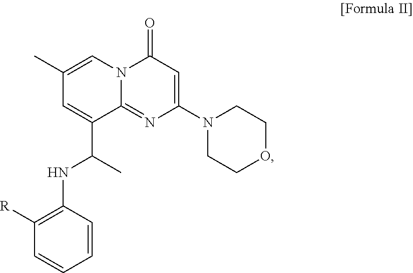
wherein R is H or CO₂H.

5. The method of claim 3, wherein the phosphoinositide 3-kinase beta inhibitor is a compound of formula IIIa or a pharmaceutically acceptable salt thereof:

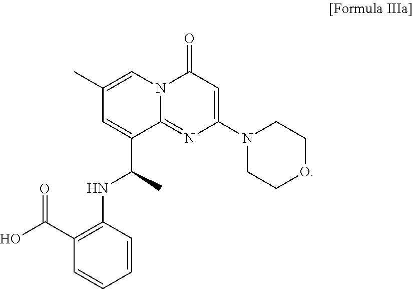 [SMILES: Cc1cc([C@@H](C)Nc2ccccc2C(=O)O)c2nc(N3CCOCC3)cc(=O)n2c1]

6. The method of any preceding claim, wherein the thrombolytic agent is tissue plasminogen activator.

7. The method of any preceding claim, which is a method of treating a subject suffering from or at risk of suffering from damage to the endothelial cells of the vasculature.

8. The method of any preceding claim, wherein the subject is suffering from thrombosis in the subject's arteries, veins and/or microvasculature.

9. The method of any preceding claim, which is a method of treating stroke or acute myocardial infarction.

10. The method of any preceding claim, wherein the subject has received or is considered appropriate to receive a thrombectomy, and/or has been treated with a stent or is about to be treated with a stent, and/or is at increased risk of symptomatic intracerebral haemorrhage (sICH), and/or has been diagnosed with intracranial atherosclerotic disease (ICAD) or as being at risk of developing ICAD.

11. The method of any preceding claim, wherein the subject has been administered a thrombolytic agent and the anti-platelet agent is administered after stopping administration of tissue plasminogen activator.

12. The method of claim 11, wherein the anti-platelet agent is administered simultaneously with an anti-coagulant.

13. The method of any preceding claim, wherein the anti-platelet agent is administered when the subject exhibits any one or more of the following symptoms:
increased vascular resistance;
a decreased blood oxygen level;
an increased level of a thrombus degradation product;
an increased level of one or more inflammatory markers;
an increased level of serum ferritin;
a decreased level of blood platelets;
a decreased level of blood clotting factors;
increased bleeding;
a requirement for mechanical ventilation.

14. The method of any preceding claim, wherein administration of the anti-platelet agent is stopped when the subject exhibits any one or more of the following symptoms:
decreased vascular resistance;
an increased blood oxygen level;
a decreased level of a thrombus degradation product;
a decreased level of one or more inflammatory markers;
a decreased level of serum ferritin;
an increased level of blood platelets;
an increased level of blood clotting factors;
decreased bleeding;
no further requirement for mechanical ventilation.

15. The method of claim 13 or claim 14, wherein
the vascular resistance is pulmonary vascular resistance;
the decreased blood oxygen level requires the subject to be administered oxygen via a mask or a mechanical ventilator or via intubation;
the thrombus degradation product is a D-dimer; and/or
the one or more inflammatory markers comprise C-reactive protein, plasma viscosity and erythrocyte sedimentation rate.

16. The method of any one of claims 13 to 15, wherein treatment with the anti-platelet agent is started within 12 hours of the subject exhibiting any one or more of said symptoms.

17. The method of any preceding claim, which is a prophylactic method of treatment.

18. The method of claim 17, wherein the anti-platelet agent is administered to the subject simultaneously, separately or sequentially with an anti-coagulant.

19. The method of any preceding claim, wherein the anti-platelet agent is a compound of formula IIIa or a pharmaceutically acceptable salt thereof: 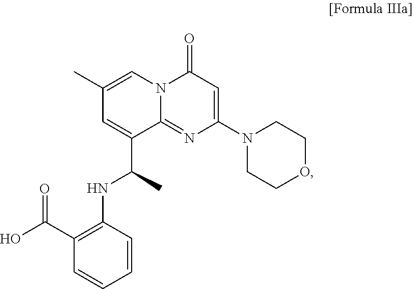 [SMILES: Cc1cc([C@@H](C)Nc2ccccc2C(=O)O)c2nc(N3CCOCC3)cc(=O)n2c1]
which is administered to the subject at a dose of from 30 mg to 185 mg.

20. The method of claim 19, wherein the compound of formula IIIa is administered to the subject at a dose of 121.5 mg.

21. The method of claim 19 or claim 20, wherein the compound of formula IIIa is administered to the subject intravenously.

22. The method of claim 21, wherein the compound of formula IIIa is administered to the subject intravenously for 3 hours.

23. The method of any preceding claim, wherein the subject has been administered or is being administered tissue plasminogen activator at a dose of from 0.6 to 0.9 mg/kg intravenously.

24. Use of an anti-platelet agent in the manufacture of a medicament for treating thrombosis or a disease or condition resulting from or associated with thrombosis in a subject in need thereof, wherein the medicament is prepared for simultaneous, sequential or separate administration with a thrombolytic agent and/or an anti-coagulant.

25. An anti-platelet agent for use in treating thrombosis or a disease or condition resulting from or associated with thrombosis in a subject in need thereof, wherein the anti-platelet agent is for simultaneous, sequential or separate administration with a thrombolytic agent and/or an anti-coagulant.

26. The use of claim 24, or the anti-platelet agent for use of claim 25, modified by the features of any one of claims 2 to 23.

27. A method of improving the efficacy of a thrombolytic agent administered to a subject in need thereof, the method comprising simultaneously, separately or sequentially administering to the subject an anti-platelet agent.

28. A method of reducing risk of bleeding in a subject receiving a thrombolytic agent and/or an anti-coagulant, the method comprising simultaneously, separately or sequentially administering to the subject an anti-platelet agent.

29. A method of inhibiting re-thrombosis in a subject receiving a thrombolytic agent and/or an anti-coagulant, the method comprising simultaneously, separately or sequentially administering to the subject an anti-platelet agent.

30. A method of inhibiting re-thrombosis in a subject who has received or is considered appropriate to receive a thrombectomy, and/or who has been treated with a stent or is about to be treated with a stent, and/or who is at increased risk of symptomatic intracerebral haemorrhage (sICH), and/or who has been diagnosed with intracranial atherosclerotic disease (ICAD) or as being at risk of developing ICAD, the method comprising administering to the subject an anti-platelet agent, wherein the anti-platelet agent is administered to the subject simultaneously, separately or sequentially with a thrombolytic agent and/or an anticoagulant.

31. The method of any one of claims 27 to 30, wherein the anti-platelet agent is a phosphoinositide 3-kinase beta inhibitor.

32. The method of claim 31, wherein the phosphoinositide 3-kinase beta inhibitor is a compound of formula II or a pharmaceutically acceptable salt thereof: 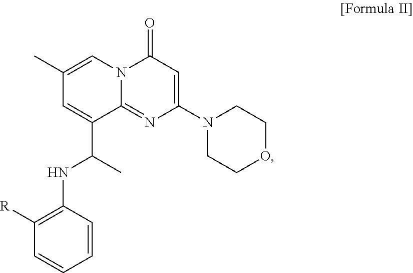
wherein R is H or CO₂H.

33. The method of claim 31, wherein the phosphoinositide 3-kinase beta inhibitor is a compound of formula IIIa or a pharmaceutically acceptable salt thereof:

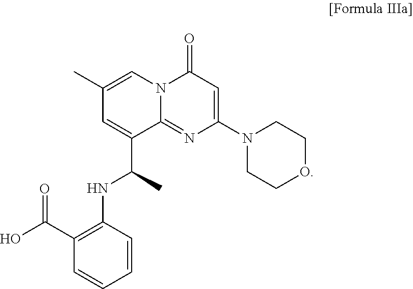 [SMILES: Cc1cc([C@@H](C)Nc2ccccc2C(=O)O)c2nc(N3CCOCC3)cc(=O)n2c1]

34. The method of any one of claims 27 to 33, wherein the thrombolytic agent is tissue plasminogen activator.

35. The method of any one of claims 27 to 34, wherein the method comprises the simultaneous, sequential or separate administration of an anti-platelet agent, a thrombolytic agent and an anti-coagulant.
